SEQUENCE LISTING (8 sequences)

SEQ ID NO: 1 - DNA, 54 bp, Artificial Sequence
  Description of Artificial Sequence Synthetic oligonucleotide
ggaaaggacgaaacaccgtgcgtggagcggcctcgcggttttagagctagaaat

SEQ ID NO: 2 - DNA, 54 bp, Artificial Sequence
  Description of Artificial Sequence Synthetic oligonucleotide
ggaaaggacgaaacaccgcacaaaagaccacaccgtcgttttagagctagaaat

SEQ ID NO: 3 - DNA, 53 bp, Artificial Sequence
  Description of Artificial Sequence Synthetic oligonucleotide
ctaaaaccgcgaggccgctccacgcacggtgtttcgtcctttccacaagatat

SEQ ID NO: 4 - DNA, 53 bp, Artificial Sequence
  Description of Artificial Sequence Synthetic oligonucleotide
ctaaaacgacggtgtggtcttttgtgcggtgtttcgtcctttccacaagatat

SEQ ID NO: 5 - DNA, 52 bp, Artificial Sequence
  Description of Artificial Sequence Synthetic primer
tcgtcggcagcgtcagatgtgtataagagacagtgcactgccggtaactctg

SEQ ID NO: 6 - DNA, 56 bp, Artificial Sequence
  Description of Artificial Sequence Synthetic primer
tcgtcggcagcgtcagatgtgtataagagacaggcagtgctaaaatacatcagggt

SEQ ID NO: 7 - DNA, 54 bp, Artificial Sequence
  Description of Artificial Sequence Synthetic primer
gtctcgtgggctcggagatgtgtataagagacagctgcccagcactctagaacc

SEQ ID NO: 8 - DNA, 54 bp, Artificial Sequence
  Description of Artificial Sequence Synthetic primer
gtctcgtgggctcggagatgtgtataagagacagagctgtgaagacaacgcaga

(19) United States
(12) Patent Application Publication
(10) Pub. No.: US 2021/0340501 A1
(43) Pub. Date: Nov. 4, 2021

(54) Methods to Control Viral Infection in Mammalian Cells

(71) Applicants: The Regents of the University of California, Oakland, CA (US); The Food and Drug Administration, Silver Spring, MD (US)

(72) Inventors: Nathan Lewis, La Jolla, CA (US); Wan-Tien Chiang, La Jolla, CA (US); Montserrat Puig, Silver Spring, MD (US); Yaqin Zhang, Silver Spring, MD (US); Amy Rosenberg, Silver Spring, MD (US)

(21) Appl. No.: 17/270,501

(22) PCT Filed: Aug. 27, 2019
(86) PCT No.: PCT/US19/48361

§ 371 Date: Feb. 23, 2021

Related U.S. Application Data
(60) Provisional application No. 62/723,233, filed on Aug. 27, 2018.

(51) Int. Cl. C12N 5/071

(52) U.S. Cl. CPC C12N 5/0682; C12N 2501/24; C12N 2501/998; C12N 2501/90; C12N 2501/999; C12N 2501/50

(57) ABSTRACT

A significant interferon (IFN) response is induced following treatment of CHO cells with exogenously-added type I IFN or poly I:C. Treatment of the CHO cells with poly I:C prior to infection limited the cytopathic effect from Vesicular stomatitis virus (VSV), Encephalomyocarditis vims (EMCV), and Reovirus-3 vims (Reo) in a STAT1-dependent manner By knocking out two upstream repressors of STAT1: Gfi1 and Trim 24, the engineered CHO cells exhibited increased resistance to virus contaminations. Thus, omics-guided engineering of mammalian cell culture can be deployed to increase safety in biotherapeutic protein production.

### CROSS REFERENCE TO RELATED APPLICATIONS

This application claims the priority benefit of U.S. Provisional Application No. 62/723,233 filed Aug. 27, 2018, which application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Aug. 27, 2019, is named 24978-0516_SL.txt and is 2,156 bytes in size.

### TECHNICAL FIELD

The present invention relates to methods to control viral infection of mammalian cells.

### BACKGROUND

Chinese hamster ovary (CHO) cells are extensively used to produce biopharmaceuticals (Walsh 2014) for numerous reasons. Though one advantage is their reduced susceptibility to many human virus families (Berting et al. 2010; Poiley et al. 1991; Weiebe et al. 1989), there have been episodes of animal viral contamination of biopharmaceutical production runs, mostly from trace levels of viruses in raw materials. These infections have led to expensive decontamination efforts and threatened the supply of critical drugs (Dinowitz et al. 1992; Garnick 1998; Nims 2006). Viruses that have halted production of valuable therapeutics include RNA viruses such as Cache Valley virus (Nims 2006), Epizootic hemorrhagic disease virus (Rabenau et al. 1993), Reovirus (Nims 2006) and Vesivirus 2117 (Bethencourt 2009). Recently, a strategy was proposed to inhibit infection of CHO cells by a limited number of rodent viruses by engineering glycosylation (Mascarenhas et al. 2017), there is a need to understand the mechanisms by which CHO cells are infected and how the cells can be universally engineered to enhance their viral resistance (Merten 2002).

Many studies have investigated the cellular response to a diverse range of viruses in mammalian cells, and detailed the innate immune responses that are activated upon infection. For example, type I interferon (IFN) responses play an essential role in regulating the innate immune response and inhibiting viral infection (Perry et al. 2005; Sadler and Williams 2008; Schoggins and Rice 2011; Taniguchi and Takaoka 2002) and can be induced by treatment of cells with poly I:C (Green and Montagnani 2013; Pantelic et al. 2005; Plant et al. 2005). However, the detailed mechanisms of virus infection and the antiviral response in CHO cells remain largely unknown. Understanding the role of type I IFN-mediated innate immune responses in CHO cells could be invaluable for developing effective virus-resistant CHO bioprocesses. Fortunately, the application of recent genome sequencing (Chen et al. 2017; Lewis et al. 2013; Rupp et al. 2018; van Wijk et al. 2017; Vishwanathan et al. 2016; Xu et al. 2011; Yusufi et al. 2018) and RNA-Seq tools can now allow the analysis of complicated cellular processes in CHO cells (Fomina-Yadlin et al. 2015; Hsu et al. 2017; Vishwanathan et al. 2015; Wang et al. 2009; Yuk et al. 2014), such as virus infection.

### SUMMARY OF THE INVENTION

The present invention provides, in embodiments, a method of inhibiting viral infection in a biological sample comprising administering to the sample an effective amount of: a) a type I interferon or poly I:C; b) a compound activating an innate immune response in the sample; c) a compound suppressing expression of Gfi1, Trim24 and/or Cb1 in the sample; and/or d) a compound activating expression of IRF7, IRF3, STAT1, STAT3, NFATC2, IRF5, STAT4, IRF9, IRF8, NFKB1, TP53, JUN and/or EBF1 in the sample. Activation or suppression of additional genes provided herein are also contemplated in all methods of the present invention.

In embodiments, the biological sample is a cell culture. In embodiments, biological sample comprises mammalian cells. In embodiments, the biological sample comprises CHO cells. In embodiments, the method is conducted in a biopharmaceutical manufacturing process.

The present invention provides, in embodiments, a non-naturally occurring mammalian cell culture comprising cells genetically modified for suppressed expression of Gfi1, Trim24 and/or Cb1, or activated expression of IRF7, IRF3, STAT1, STAT3, NFATC2, IRF5, STAT4, IRF9, IRF8, NFKB1, TP53, JUN and/or EBF1, as compared to wild-type cells of the same mammalian species.

The present invention provides, in embodiments, a method of producing a biopharmaceutical protein from a mammalian cell culture, comprising culturing mammalian cells having non-naturally occurring genetically suppressed expression of Gfi1, Trim24 and/or Cb1, or genetically activated expression of IRF7, IRF3, STAT1, STAT3, NFATC2, IRF5, STAT4, IRF9, IRF8, NFKB1, TP53, JUN and/or EBF1, or both, as compared to wild-type cells of the same mammalian species; and isolating a protein of interest from the cultured cells.

The present invention provides, in embodiments, a method of treating or preventing viral infection in a mammalian cell comprising administering to the cell an effective amount of: a) a type I interferon or poly I:C; b) a compound activating an innate immune response in the sample; c) a compound suppressing expression of Gfi1, Trim24 and/or Cb1 in the sample; and/or d) a compound activating expression of IRF7, IRF3, STAT1, STAT3, NFATC2, IRF5, STAT4, IRF9, IRF8, NFKB1, TP53, JUN and/or EBF1 in the sample.

The present invention provides, in embodiments, a method for increasing virus infectivity in a mammalian cell comprising increasing expression of Gfi1, Trim24 and/or Cb1, or decreasing expression of IRF7, IRF3, STAT1, STAT3, NFATC2, IRF5, STAT4, IRF9, IRF8, NFKB1, TP53, JUN and/or EBF1 in the cell. In embodiments, the method further comprises isolating virus or viral particles from the cell. In embodiments, genetic material is delivered to the sample by viral transduction to increase or decrease expression of said gene.

The present invention provides, in embodiments, a non-naturally occurring mammalian cell culture comprising mammalian cells having genetically activated expression of Gfi1, Trim24 and/or Cb1, or genetically suppressed expression of IRF7, IRF3, STAT1, STAT3, NFATC2, IRF5, STAT4, IRF9, IRF8, NFKB1, TP53, JUN and/or EBF1, as compared to wild-type cells of the same mammalian species.

### BRIEF DESCRIPTION OF THE DRAWINGS

FIGS. 1A-1E show RNA viruses induce cytopathic effects on CHO-K1 cells. (FIG. 1A) Cytopathic effect of the three RNA viruses on CHO cells upon 30 h (VSV), 54 h (EMCV) or 78 h (Reo) of infection. Fold change in (FIG. 1B) IFNβ and (FIG. 1C) Mx1 gene expressions in CHO cells infected with the three RNA viruses compared to uninfected cells at the same time points. FIG. 1D shows several pathways and processes were enriched for differentially expressed genes following viral infection (m vs. Vm). FIG. 1E shows activated (red) or repressed (blue) upstream regulators following virus infection.

FIGS. 2A-2E show innate immunity genes in CHO cells are activated by poly I:C. (FIG. 2A) IFN-stimulated transcription was increased in cells treated with poly I:C/LyoVec for 24 h, but not with other TLR ligands engaging TLR9, TLR4 or TLR7/8. (FIG. 2B) Poly I:C triggered STAT1 phosphorylation in a dose dependent manner, and (FIG. 2C) the levels of STAT2 phosphorylation and Mx1 protein expression were comparable to those triggered by IFNα2c. (FIG. 2D) Several pathways and processes were enriched for differentially expressed genes following poly I:C treatment (m vs. p). (FIG. 2E) Upstream regulators that are activated (red greyscales) or repressed (blue greyscales) following poly I:C treatment.

FIGS. 3A-3E show Poly I:C pre-treatment prevents virus infection of VCV, EMCV, and Reo. (FIGS. 3A-3C) Cell morphology (left panels) and cytopathic effect measured by crystal violet staining (right panels) of virus-infected CHO cells (Note that panels a, b, c and d corresponds to ‘m’ ‘p’, ‘Vm’ and ‘Vp’, respectively); (FIG. 3D) the enriched down-stream pathways under condition of Vm vs. Vp using RNA-Seq data. (FIG. 3E) The top 35 upstream regulators that are activated or repressed.

FIGS. 4A-4B show a STAT1-dependent regulatory network controls viral resistance (VSV and EMCV) in CHO cells. A STAT1-dependent regulatory network induced by the pretreatment of poly I:C leads to the inhibition of (FIG. 4A) VSV and (FIG. 4B) EMCV replication in CHO cells, based on the comparison of Vm and Vp RNA-Seq. The greyscales denote the states inferred from the RNA-Seq data. For example, the blue greyscales of TRIM24 means that TRIM24 activity is suppressed, based on the differential expression of genes that are regulated by TRIM24.

FIG. 5 shows enhanced virus resistance through genetic engineering on the repressors of STAT1. Schematic view of the genetic engineering approach in improving virus resistibility in CHO cells by knocking out repressors of STAT1.

FIGS. 6A-6C show RNA-Seq results of the Gfi1 and/or Trim24 KO engineered CHO cells. Gfi1 and Trim24 were knocked out compared to the control (susceptible) cells. Transcriptional regulatory networks were identified using IPA upstream regulatory analysis (FIG. 6A), in which the innate immunity regulatory network (JAK-STAT network) is indicated by the arrow. Transcriptional factors of the identified JAK-STAT regulatory network in the knocked down cells (FIG. 6B) and the activation of immune functions following Gfi1 and/or Trim24 genetic engineering were illustrated (FIG. 6C).

FIGS. 7A-7C show viral resistance of the Gfi1 and/or Trim24 engineered CHO cells. Gfi1 and Trim24 were knocked out and tested for resistance to EMCV and Reo-3 virus infection compared to the control (susceptible) cells (see details in Materials and methods). Cell density and viability was followed up for one week post infection (p.i.) for Gfi1 single knockout cells (FIG. 7A), Trim24 single knockout cells (FIG. 7B) and Gfi1 and Trim24 double knockout cells (FIG. 7C). To assess robustness of the observed viral resistances in EMCV and Reo-3 virus infection, the reproducibility analysis was conducted for EMCV (three replicates) and Reo-3 (two replicates) virus. Susceptible CHO cell lines were used as positive controls for EMCV and Reo-3 virus infections during the first seven days (FIGS. 17A-17B). Resistant cultures were passaged and followed up for an additional week (FIGS. 18A-18C).

FIG. 8 shows pretreatment of the cell culture with type I IFN protein limits VSV infection. Cells were cultured with the indicated concentration of human or murine IFN protein for 24 h prior to infect with VSV, which was serially diluted (1:10). Last row includes cells infected with VSV but not pretreated with IFN (6 wells) or non-infected cells (5 wells). The plate shows results from one experiment representative of 2, in which Hu-IFNα standard was used at 1000 IU/ml and gave comparable results.

FIGS. 9A-9B show enrichment strength of the interferon-alpha response. (FIG. 9A) Interferon-alpha response in the comparisons of m vs. Vm and Vm vs. Vp. (FIG. 9B) Time effects on the Interferon-alpha response induced by poly I:C on non-infected cultures. The ‘interferon-alpha response’ is a hallmark gene set of the gene set enrichment analysis (GSEA). The enrichment strength describes the leading-edge subset of a gene set (i.e., the interferon-alpha response in this study) (Subramanian et al. 2005). If the gene set is entirely within the first N positions in the ranked differentially expressed gene list, then the signal strength is maximal or 100%. If the gene set is spread throughout the list, then the signal strength decreases towards 0%.

FIG. 9A shows that the enrichment strength (65%, see EXAMPLE 2) of ‘interferon alpha response’ from the comparison of untreated media and Reo infected CHO cells (m vs. Vm) is smaller than those from the comparison of both virus presenting and poly I:C pretreated media (Vm vs. Vp; 77% and 77% for VSV and EMCV, respectively), which suggests that Reo-induced interferon alpha response might be insufficient for CHO cells limiting Reo infection. Indeed, Reo has been known to inhibit the type I IFN response using different strategies (Sherry 2009), such as modulation of cell RNA sensors (RIG-I and MDA5) and transcription factors (IRF3 and NF-kB) involved in induction of IFN. In consistent with our results, the IRF3 (z score=4.96 and p-value<0.05; FIG. 1E) and NFkB pathways (p-value=1.12×10⁻² and NES=2.22) have been observed to be activated in the comparison of m vs. Vm. While the underling mechanism of how these RNA viruses evade the (innate) immune system is still unclear, these data substantiate the inability of CHO cells to elicit protective anti-viral mechanisms by not mounting an effective protective (type I IFN) response. However, these data suggest that viral infection could likely be limited by further inducing IFN pathways.

FIG. 9B further demonstrates that temporal difference might be another factor accounting for the variations of type I interferon response. Indeed, we observed the enrichment strength of ‘interferon alpha response’ in the comparison of untreated cells and poly I:C pretreated cells (m vs. p) are different (73%, 70% and 78% for 30, 54 and 78 h, respectively). These differences might also result in the different magnitudes of downstream pathway/hallmark responses (FIG. 2D) and upstream regulator expression variations (FIG. 2E) across the different batches of samples that were collected from different time points.

FIGS. 10A-10B. IFNβ and Mx1 gene expression kinetics by poly I:C. Changes in RNA transcript levels of anti-viral genes IFNβ (FIG. 10A) and Mx1 (FIG. 10B) in CHO cells treated with poly I:C (black squares) compared to untreated cultures (open circles) over time.

FIGS. 11A-11B. Poly I:C pre-treatment of CHO cells protect against viral infection through the IFNβ-mediated pathway. (FIG. 11A) Poly I:C can induce effective anti-viral mechanisms in CHO cells. (FIG. 11B) IFNβ plays a protective role in the VSV infection, as treatment with anti-IFNβ neutralizing Ab abolishes the protective effect of poly I:C treatment.

FIG. 12. Differential induction of antiviral genes (Mx1 and IITMP3) by poly I:C and VSV or EMCV as opposed to Reo. Expression levels of Mx1 and IITMP3 were measured by Taqman real-time PCR (qPCR) and RNA-Seq. The x-axis represents the comparisons of the two indicated culture conditions. The y-axis denotes the log₂ values of fold change (log₂(FC)). The black bars represent the values of the differential fold change were calculated from the qPCR data, and the white bars denote the values of the differential fold change were calculated from the RNA-Seq data using the R package of DESeq2.

FIGS. 13A-13B. Up-regulated DEGs present in m vs. Vp and m vs. p but not in m vs. Vm. (FIG. 13A) Venn diagram of up regulated genes across different comparisons and the enriched KEGG pathways for the 30 DEGs that present with poly I:C treatment but not in Reo infection. (FIG. 13B) Example of the most enriched pathway: “antigene processing and presentation” for the 30 DEGs. Note that, the criteria for identifying up regulated DEGs are: adjust p-value<0.05 and fold change>1.5 in the differential expressed genes test using DESeq2.

FIG. 14. Poly I:C pretreatment activates STAT1-dependent network in CHO cells. A STAT1-dependent regulatory network induced by the pretreatment of poly-I:C leads to several immune related responses activated in CHO cells, based on the comparison of m and p RNA-Seq. The greyscales denote the states inferred from the RNA-Seq data. For example, the blue greyscale of TRIM24 means that TRIM24 activity is suppressed, based on the differential expression of genes that are regulated by TRIM24.

FIGS. 15A-15B. NFATC2-dependent network in inducing STAT1 for inhibiting infection of mammalian cells. (FIG. 15A) m vs. Vm. (FIG. 15B) m vs. p. Note that, the six genes (IL15, NFKB1Z, IRF1, IL18, PML and REL) that are different in these two networks are highlighted in the green greyscales dashed circles.

FIGS. 16A-16B. IRF3-dependent network inducing STAT1 for the inhibition of viral infection. (FIG. 16A) m vs. Vm. (FIG. 16B) m vs. p. Note that, the three genes (DHX58, IL15 and IFIH1) that are different in these two networks are highlighted in the green dashed circles.

FIGS. 17A-17B. Positive controls of susceptible CHO cell lines in the EMCV and Reo-3 virus infections. Susceptible CHO cell lines were used as positive controls for EMCV (FIG. 17A) and Reo-3 (FIG. 17B) virus infections (see FIG. 5) during the first seven days.

FIGS. 18A-18C. Long term culture of virus infection assay. Resistant cultures were passaged and followed up for an additional week for Gfi1 and Trim24 double knockout cells (FIG. 18A), Gfi1 single knockout cells (FIG. 18B) and Trim24 single knockout cells (FIG. 18C).

FIGS. 19A-19B. Negative regulatory scores of STAT1 upstream regulators. (FIG. 19A) Negative regulatory score of STAT1 upstream regulators in the comparison of m (Media) vs. p (poly I:C treated media). (FIG. 19B) Negative regulatory score of STAT1 upstream regulators in the comparison of Vm (Virus+Media) vs. Vp (Virus+poly I:C treated media).

### DETAILED DESCRIPTION

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

### Definitions

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or mammalian animal subject to be treated.

As used herein the term “pharmaceutical composition” refers to a pharmaceutical acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to age-related eye diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent. As used herein, and unless otherwise specified, the term “subject” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

The term “antibody” as used herein encompasses monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isoforms of interest. The term “antibody fragments” comprise a portion of a full length antibody, generally the antigen binding or variable region thereof. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art.

The invention may also refer to any oligonucleotides (antisense oligonucleotide agents), polynucleotides (e.g. therapeutic DNA), ribozymes, DNA aptamers, dsRNAs, siRNA, RNAi, and/or gene therapy vectors. The term “antisense oligonucleotide agent” refers to short synthetic segments of DNA or RNA, usually referred to as oligonucleotides, which are designed to be complementary to a sequence of a specific mRNA to inhibit the translation of the targeted mRNA by binding to a unique sequence segment on the mRNA. Antisense oligonucleotides are often developed and used in the antisense technology. The term “antisense technology” refers to a drug-discovery and development technique that involves design and use of synthetic oligonucleotides complementary to a target mRNA to inhibit production of specific disease-causing proteins. Antisense technology permits design of drugs, called antisense oligonucleotides, which intervene at the genetic level and inhibit the production of disease-associated proteins. Antisense oligonucleotide agents are developed based on genetic information.

As an alternative to antisense oligonucleotide agents, ribozymes or double stranded RNA (dsRNA), RNA interference (RNAi), and/or small interfering RNA (siRNA), can also be used as therapeutic agents for regulation of gene expression in cells. As used herein, the term “ribozyme” refers to a catalytic RNA-based enzyme with ribonuclease activity that is capable of cleaving a single-stranded nucleic acid, such as an mRNA, to which it has a complementary region. Ribozymes can be used to catalytically cleave target mRNA transcripts to thereby inhibit translation of target mRNA. The term “dsRNA,” as used herein, refers to RNA hybrids comprising two strands of RNA. The dsRNAs can be linear or circular in structure. The dsRNA may comprise ribonucleotides, ribonucleotide analogs, such as 2′-O-methyl ribosyl residues, or combinations thereof. The term “RNAi” refers to RNA interference or post-transcriptional gene silencing (PTGS). The term “siRNA” refers to small dsRNA molecules (e.g., 21-23 nucleotides) that are the mediators of the RNAi effects. RNAi is induced by the introduction of long dsRNA (up to 1-2 kb) produced by in vitro transcription, and has been successfully used to reduce gene expression in variety of organisms. In mammalian cells, RNAi uses siRNA (e.g. 22 nucleotides long) to bind to the RNA-induced silencing complex (RISC), which then binds to any matching mRNA sequence to degrade target mRNA, thus, silences the gene.

The present invention provides, in embodiments, a method of inhibiting viral infection in a biological sample comprising administering to the sample an effective amount of: a) a type I interferon or poly I:C; b) a compound activating an innate immune response in the sample; c) a compound suppressing expression of Gfi1, Trim24 and/or Cb1 in the sample; and/or d) a compound activating expression of IRF7, IRF3, STAT1, STAT3, NFATC2, IRF5, STAT4, IRF9, IRF8, NFKB1, TP53, JUN and/or EBF1 in the sample. Activation or suppression of additional genes provided herein are also contemplated in all methods of the present invention.

In embodiments, the biological sample is a cell culture. In embodiments, biological sample comprises mammalian cells. In embodiments, the biological sample comprises CHO cells. In embodiments, the method is conducted in a biopharmaceutical manufacturing process. In embodiments, the compound suppresses expression of Gfi1, Trim24 and/or Cb1 in the sample. In embodiments, the compound activates expression of IRF7, IRF3, STAT1, STAT3, NFATC2, IRF5, STAT4, IRF9, IRF8, NFKB1, TP53, JUN and/or EBF1 in the sample. In embodiments, the virus is VSV, EMCV, REO, or a RNA virus. The virus can also be a DNA virus.

The present invention provides, in embodiments, that the compound for activating/increasing genetic expression or suppressing/decreasing genetic expression can be a nucleic acid. The nucleic acid can be transduced into the cell by methods well-known to those of skill in the art. In embodiments, the compound for activating/increasing genetic expression or suppressing/decreasing genetic expression can be a small molecule, transcription factor, microRNA (miRNA), small interfering RNA (siRNA), RNAi, Zinc Finger Nucleases/Peptides, TALENS, antibody, aptamer, or other functional agent. Non-coding nucleic acids can also be used as a compound for modulating the expression of genes: antisense oligonucleotides, antisense DNA or RNA, triplex-forming oligonucleotides, catalytic nucleic acids (e.g. ribozymes), nucleic acids used in co-suppression or gene silencing, or similar systems to activate/increase or suppress/decrease the genetic expression. Well-known genetic engineering techniques such as site-directed knock-out (KO), knock-in (KI), knock-down (KD), gene mutation, gene transfection, CRISPR activation, CRISPR inhibition, CRISPR/Cas9, and other gene editing systems can also be used as compounds to modify genes and expressional levels as described herein. Compounds to modify expression can include poly I:C or drugs that activate/increase or suppress/decrease the innate immune response.

The present invention provides, in embodiments, a non-naturally occurring mammalian cell culture comprising cells genetically modified for suppressed expression of Gfi1, Trim24 and/or Cb1, and/or activated expression of IRF7, IRF3, STAT1, STAT3, NFATC2, IRF5, STAT4, IRF9, IRF8, NFKB1, TP53, JUN and/or EBF1, as compared to wild-type cells of the same mammalian species.

The present invention provides, in embodiments, a method of producing a biopharmaceutical protein from a mammalian cell culture, comprising culturing mammalian cells having non-naturally occurring genetically suppressed expression of Gfi1, Trim24 and/or Cb1, and/or genetically activated expression of IRF7, IRF3, STAT1, STAT3, NFATC2, IRF5, STAT4, IRF9, IRF8, NFKB1, TP53, JUN or EBF1 or TP53 or JUN and/or EBF1, as compared to wild-type cells of the same mammalian species; and isolating a protein of interest from the cultured cells.

The present invention provides, in embodiments, that the biological sample comprises CHO cells. In embodiments, the cells have suppressed expression of Gfi1, Trim24 and/or Cb1. In embodiments, the cells have activated expression of IRF7, IRF3, STAT1, STAT3, NFATC2, IRF5, STAT4, IRF9, IRF8, NFKB1, TP53, JUN and/or EBF1.

The present invention provides, in embodiments, a method of treating or preventing viral infection in a mammalian cell comprising administering to the cell an effective amount of: a) a type I interferon or poly I:C; b) a compound activating an innate immune response in the sample; c) a compound suppressing expression of Gfi1, Trim24 and/or Cb1 in the sample; and/or d) a compound activating expression of IRF7, IRF3, STAT1, STAT3, NFATC2, IRF5, STAT4, IRF9, IRF8, NFKB1, TP53, JUN and/or EBF1 in the sample.

The present invention provides, in embodiments, a method to block viral infection in mammalian cells in vivo having genetically or chemically decreased activity of Gfi1, Trim24 and/or Cb1, and/or genetically or chemically increased expression and/or activity of IRF7, IRF3, STAT1, STAT3, NFATC2, IRF5, STAT4, IRF9, IRF8, NFKB1, TP53, JUN and/or EBF1, as compared to wild-type cells of the same mammalian species.

The present invention provides, in embodiments, a method for increasing virus infectivity in a mammalian cell comprising increasing expression of Gfi1, Trim24 and/or Cb1, and/or decreasing expression of IRF7, IRF3, STAT1, STAT3, NFATC2, IRF5, STAT4, IRF9, IRF8, NFKB1, TP53, JUN and/or EBF1 in the cell. In embodiments, the method further comprises isolating virus or viral particles from the cell. In embodiments, genetic material is delivered to the sample by viral transduction to increase or decrease expression of said gene.

The present invention provides, in embodiments, a non-naturally occurring mammalian cell culture comprising mammalian cells having genetically activated expression of Gfi1, Trim24 or Cb1, and/or genetically suppressed expression of IRF7, IRF3, STAT1, STAT3, NFATC2, IRF5, STAT4, IRF9, IRF8, NFKB1, TP53, JUN and/or EBF1, as compared to wild-type cells of the same mammalian species.

These and other embodiments of the invention will be apparent to one of skill in the art upon a review of the present Specification.

### Example 1

### Materials and Methods

### CHO-K1 Cells and RNA Viruses

The susceptibility of CHO-K1 cells to viral infection has been previously reported (Berting et al. 2010). Since infectivity was demonstrated for viruses of a variety of families (harboring distinct genomic structures), the following RNA viruses were selected from three different families to be used as prototypes: Vesicular stomatitis virus (VSV, ATCC VR-1238), Encephalomyocarditis virus (EMCV, ATCC VR-129B), and Reovirus-3 virus (Reo, ATCC VR-824). Viral stocks were generated in susceptible Vero cells as per standard practices using DMEM (Dulbecco's Modified Eagle's medium) supplemented with 10% FBS, 2 mM L-glutamine, 100 U/ml penicillin and 100 μg/ml streptomycin (DMEM-10). Viral stocks were tittered by tissue culture infectious dose 50 (TCID₅₀) on CHO-K1 cells and used to calculate the multiplicity of infection in the experiments (Table 1).

### Virus Infection and Innate Immune Modulator Treatment.

Cells were seeded in cell culture plates (3×10⁵ and 1.2×10⁶ cells/well in 96-well and 6-well plates, respectively) and grown overnight in RPMI-1040 supplemented with 10% FBS, 2 mM L-glutamine, 100 U/ml penicillin and 100 ng/ml streptomycin, 10 mM Hepes, lx non-essential amino acids and 1 mM sodium pyruvate (RPMI-10). IFNα/f3 and innate immune modulators (LPS (TLR4) (Calbiochem), CpG-oligodeoxynucleotide (ODN) D-ODN, (Puig et al. 2012) and ODN-1555, (TLR9) (custom-synthesized at the Center for Biologics Evaluation and Research facility, FDA), imidazoquinoline R837 (TLR7/8) (Sigma) and poly I:C-Low molecular weight/LyoVec (polyinosinic-polycytidylic acid) (poly I:C) (Invivogen) were added to the cultures 16-24 h prior to virus infection, at the concentrations indicated in the figures. Viral infection was performed by adding virus suspensions to the cell monolayers at the indicated MOI in serum-free media and incubate at 37° C. 5% CO₂ for 2 h. Cell cultures were washed twice to discard unbound virus and further incubated at 37° C. for 30 h (VSV), 54 h (EMCV) or 78 h (Reo) (unless otherwise indicated in the figures). The cell harvesting time was established based on appearance of cytopathic effect in approximately 50% of the cell monolayer. Cytopathic effect was visualized by crystal violet staining as per standard practices. Infection/poly 1:C experiments were repeated twice, independently. In each replicate CHO cells were cultured as poly I:C untreated—uninfected (media control, m), poly I:C treated—uninfected (p), poly I:C untreated—virus infected (Vm) and poly I:C treated—virus infected (Vp). The antibodies and cytokines used as innate immune modulators were anti-STAT1 and pSTAT2 antibodies (Becton Dickinson), neutralizing anti-IFNβ antibody (R&D), anti-Mx1 antibodies (a gift from Dr. O. Haller, Germany). Human IFNα (Avonex) and IFNβ (Roferon) are clinical grade drugs.

Western blot procedures. Cell lysates were prepared using mammalian protein extraction reagent M-PER (Thermo Fisher Scientific, Waltham, Mass.) with Protease and Halt™ phosphatase inhibitor cocktails (Thermo Fisher Scientific) using an equal number of cells per sample. Samples were analyzed by SDS-PAGE using 10-20% Tris-Glycine gels (Thermo Fisher Scientific) under reducing conditions. As a molecular weight marker, protein ladder (cat #7727S) from Cell Signaling Technology (Danvers, Mass.) was used. Nitrocellulose membranes and iBlot™ transfer system (Thermo Fisher Scientific) were used for Western Blot analysis. All other reagents for Western Blot analyses were purchased from Thermo Fisher Scientific. Membranes were blocked with nonfat dry milk (BIO-RAD, Hercules, Calif.) for 1 h followed by incubation with primary antibodies against STAT1, pSTAT1 (pY701, BD Transduction Lab, San Jose, Calif.), or Mx1 (gift from O. Haller, University of Freiburg, Freiburg, Germany) O/N at 4° C. Secondary goat anti-mouse and anti-rabbit antibodies were purchased from Santa Cruz Biotechnology. SuperSignal West Femto Maximum Sensitivity Kit (Thermo Fisher Scientific) was used to develop membranes, and images were taken using LAS-3000 Imaging system (GE Healthcare Bio-Sciences, Pittsburgh, Pa.).

### RNA Extraction, Purification, and Quality Control Procedure

Cell cultures were resuspended in RLT buffer (Qiagen) and kept at −80° C. until RNA was extracted using the RNeasy kit (Qiagen) and on-column DNAse digestion. RNA was eluted in 25 μl of DEPC water (RNAse/DNAse free); concentration and purity were tested by bioanalyzer. Total RNA levels for type I IFN related genes and viral genome were also assessed by RT-PCR. Complementary DNA synthesis was obtained from 1 μg of RNA using the High capacity cDNA RT kit (Thermo Fisher Scientific) as per manufacturer's instructions. Semi-quantitative PCR reactions (25 μl) consisted in 1/20 cDNA reaction volume, 1× Power Sybr master mix (Thermo Fisher Scientific), 0.5 μM Chinese hamster-specific primers for IFNβ, Mx1, IRF7 and IITMP3 sequences (SAbiosciences) (these genes were selected to assess type I IFN response). Eukaryotic 18S was used as a housekeeping gene and assessed in 1× Universal master mix, 18S expression assay (1:20) (Applied Biosystems) using a 1/50 cDNA reaction volume. Fold changes were calculated by the 2-ΔΔCt method.

cDNA Library Construction and Next-Generation Sequencing (RNA-Seq)

Library preparation was performed with Illumina's TruSeq Stranded mRNA Library Prep Kit High Throughput (Catalog ID: RS-122-2103), according to manufacturer's protocol. Final RNA libraries were first quantified by Qubit HS and then QC on Fragment Analyzer (from Advanced Analytical). Final pool of libraries was run on the NextSeq platform with high output flow cell configuration (NextSeq 500/550 High Output Kit v2 (300 cycles) FC-404-2004).

### RNA-Seq Quantification and Differential Gene Expression Analysis

RNA-Seq quality was assessed using FastQC. Adapter sequences and low quality bases were trimmed using Trimmomatic (Bolger et al. 2014). Sequence alignment was accomplished using STAR (Dobin et al. 2013) against the CHO genome (GCF_000419365.1_C_griseus_v1.0) with default parameters. HTSeq (Anders et al. 2015) was used to quantify the expression of each gene. Differential gene expression analysis using DESeq2 (Anders and Huber 2010). After Benjamini-Hochberg FDR correction, genes with adjusted p-values less than 0.05 and fold change greater than 1.5 were considered as differentially expressed genes (DEGs). Table 3 shows the number of identified DEGs in the three different comparisons: 1) untreated—uninfected vs. untreated—virus infected (m vs. Vm); 2) untreated—uninfected vs. poly I:C treated—uninfected (m vs. p); and 3) untreated—virus infected vs. poly I:C treated—virus infected (Vm vs. Vp).

### Genetic Engineering (Gfi1, Trim24, Gfi1/Trim24) of CHO-S Cell Lines

CHO-S cells (Thermo Fisher Scientific Cat. #A1155701) and KO clones were cultured in CD CHO medium supplemented with 8 mM L-glutamine and 2 mL/L of anti-clumping agent (CHO medium) in an incubator at 37° C., 5% CO₂, 95% humidity. Cells were transfected using FuGENE HD reagent (Promega Cat. #E2311). The day prior to transfection, viable cell density was adjusted to 8×10⁵ cells/mL in an MD6 plate well containing 3 mL CD CHO medium supplemented with 8 mM L-glutamine. For each transfection, 1500 ng Cas9-2A-GFP plasmid and 1500 ng gRNA plasmid were diluted in 75 uL OptiPro SFM. Separately, 9 uL FuGene HD reagent was diluted in 66 uL OptiPro SFM. The diluted plasmid was added to the diluted FuGENE HD and incubated at room temperature for 5 minutes and the resultant 150 μL DNA/lipid mixture was added dropwise to the cells. For viability experiments, CHO-S KO cell lines were seeded at 3×10⁶ cells in 30 ml in CHO medium and incubated at 37° C., 5% CO₂, 125 rpm for up to 7 days. Infections were conducted with EMCV and Reo-3 at the same MOI calculated in CHO-K1 cells for 2 h prior to wash cells twice to discard unbound particles. Control cell lines showing susceptibility to either virus were infected in parallel to those with Gfi1 and Trim24 gene KO.

The plasmids we used to generate Gfi, Trim24, and Gfi+Trim24 knock-out cell lines are: Plasmids 2632 (GFP_2A_Cas9), Plasmids 6016 (Gfi1-665755) and 6018 (Trim24-1009774). The Plasmids 2632 (GFP_2A_Cas9) is described in (Gray et al., 2015). The Plasmids 6016 (Gfi1-665755) and 6018 (Trim24-1009774) were constructed as described in (Ronda et al., 2014) with the following modification: sgRNA plasmid sgRNA1_C described in (Ronda et al., 2014) was used as template in the PCR reaction to generate the backbone of gRNA plasmids.

Oligos used in the cloning reaction were:

| | | | |
|---|---|---|---|
| 17229 | Gfil- | GGAAAGGACGAAACACCGTGCGTGGAGCG | SEQ ID NO: 1 |
| | 665755_gRNAfwd | GCCT CGCGGTTTTAGAGCTAGAAAT | |
| 17231 | Trim24- | GGAAAGGACGAAACACCGCACAAAAGAC | SEQ ID NO: 2 |
| | 1009774_gRNAfwd | CACACCGT CGTTTTAGAGCTAGAAAT | |
| 17325 | Gfil- | CTAAAACCGCGAGGCCGCTCCACGCACGG | SEQ ID NO: 3 |
| | 665755_gRNArev | TGTTTCGTCCTTTCCACAAGATAT | |
| 17327 | Trim24- | CTAAAACGACGGTGTGGTCTTTTGTGCGGT | SEQ ID NO: 4 |
| | 1009774_gRNArev | GTTTCGTCCTTTCCACAAGATAT | |

### Primers Used for MISEQ Analysis were

| | | | |
|---|---|---|---|
| 18484 | Gfil- | TCGTCGGCAGCGTCAGATGTGTATAAGAG | SEQ ID NO: 5 |
| | 1665755_MiSeqfwd | ACAGTGCACTGCCGGTAACTCTG | |
| 17423 | Trim24-MiSeqfwd | TCGTCGGCAGCGTCAGATGTGTATAAGAG | SEQ ID NO: 6 |
| | | ACAGGCAGTGCTAAAATACATCAGGGT | |
| 18485 | Gfil- | GTCTCGTGGGCTCGGAGATGTGTATAAGA | SEQ ID NO: 7 |
| | 1665755_MiSeqrev | GACAGCTGCCCAGCACTCTAGAACC | |
| 17519 | Trim24- | GTCTCGTGGGCTCGGAGATGTGTATAAGA | SEQ ID NO: 8 |
| | 11009774_MiSeqrev | GACAGAGCTGTGAAGACAACGCAGA | |

### Single Cell Sorting, Clone Genotyping and Expansion

Transfected cells were single cell sorted 48 hours post transfection, using a FACSJazz, based on green fluorescence with gating determined by comparison to non-transfected cells. Sorting was done into MD384 well plates (Corning Cat. #3542) containing 30 μL CD CHO medium supplemented with 8 mM L-glutamine, 1% antibiotic-antimycotic agent (Thermo Fisher Scientific Cat. #15240-062) and 1.5% HEPES buffer (Thermo Fisher Scientific Cat. #15630-056). After 15 days, colonies were transferred to an MD96 F well plate (Falcon Cat. #351172) containing 200 μL CD CHO medium supplemented with 8 mM L-glutamine, and 1% antibiotic-antimycotic. After additional two days, 50 μL cell suspension from each well was transferred to a MicroAmp Fast 96 well reaction plate (Thermo Fisher Scientific Cat. #4346907), along with 5×10⁵ wildtype control cells. The plate was centrifuged at 1000×g for 10 minutes and then the supernatant was removed by rapid inversion. Twenty μL of 65° C. QuickExtract DNA Extraction Solution (Epicentre Cat. #QE09050) was added to each well and mixed. The plate was then placed in a thermocycler at 65° C. for 15 minutes followed by 95° C. for 5 minutes. Amplicons were generated for each gene of interest per well using Phusion Hot Start II DNA Polymerase and verified to be present visually on a 2% agarose gel. Amplicons from each well had unique barcodes, allowing them to be pooled and purified using AMPure XP beads (Beckman Coulter Cat. #A63881) according to manufacturer's protocol, except using 80% ethanol for washing steps and 40 μL beads for 50 μL sample. Samples were indexed using the Nextera XT Index kit attached using 2×KAPA HiFi Hot Start Ready mix (Fisher Scientific Cat. #KK2602). AMPure XP beads were used to purify the resulting PCR products. DNA concentrations were determined with the Qubit 2.0 Fluorometer and used to pool all indices to an equimolar value and diluted to a final concentration of 10 nM using 10 mM Tris pH 8.5, 0.1% Tween 20. The average size of the final library was verified with a Bioanalyzer 2100. The amplicon library was then sequenced on an Illumina MiSeq. Insertions and deletions were identified by comparison of expected versus actual amplicon size. Clones with frameshift indels in all alleles were selected for expansion in shake flasks (shaking at 120 rpm, 25 mm throw), banking and characterization.

### Results and Discussion

### CHO-K1 Cells Fail to Prevent Infection by RNA Viruses Despite Possessing Functional Type I IFN-Inducible Anti-Viral Mechanisms.

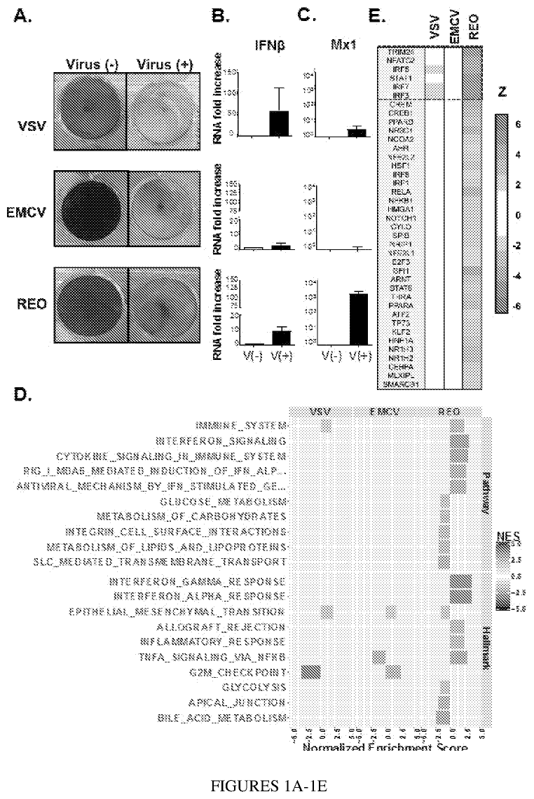

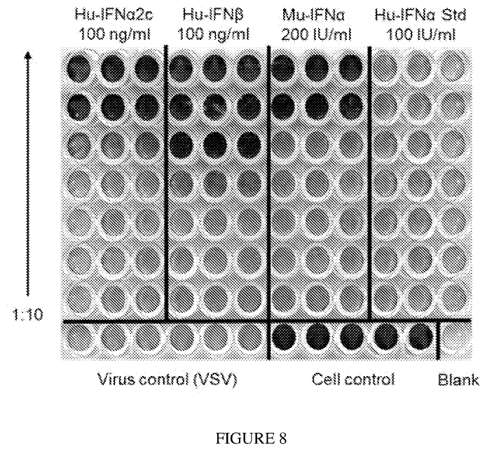

To evaluate the response of CHO cells to the three different RNA viruses (VSV, EMCV and Reo; see Table 1), CHO cells were infected and monitored for cytopathic effects and gene expression changes related to the type I IFN response (see Materials and Methods). All three viruses induced a cytopathic effect (FIG. 1A, right panels) and measured a modest increase in IFNβ transcript levels in CHO cells (FIG. 1B). Through its cellular receptor, IFNα/β can further activate downstream interferon-stimulated genes known to limit viral infection both in cell culture and in vivo (Katze et al. 2002; McNab et al. 2015; Schneider et al. 2014; Seo and Hahm 2010). Indeed, the results indicate that CHO cells have a functional IFNα/β receptor and that its activation with exogenous IFN confers resistance of CHO cells to VSV infection (see FIG. 8). Interestingly, CHO cells expressed high levels of the antiviral gene Mx1 when infected with Reo, but not VSV and EMCV (FIG. 1C). Nevertheless, the virus-induced IFN response in the host cell was insufficient to prevent cell culture destruction. These data suggest a possible inhibition of the antiviral type I IFN response that varies across viruses, as previously reported (Ahmed et al. 2003; Ng et al. 2013; Rieder and Conzelmann 2009; Sherry 2009).

To explore why the induced type I IFN failed to mount a productive antiviral response in CHO cells, RNA-Seq and pathway analysis was conducted using GSEA (see EXAMPLE 2). GSEA analysis that compared control vs. infected CHO cells (m vs. Vm) revealed the modulation of several immune-related gene sets and pathways activated by the virus (FIG. 1D). Unlike VSV and EMCV, Reo induced the ‘interferon alpha response’ and ‘RIG-I and MDA5-mediated induction of IFNα’ pathways ((p-value, NES)=(9.05×10⁻³, 3.68) and (1.12×10⁻², 2.74), respectively). These findings were consistent with observations that the reovirus genome (dsRNA) can stimulate TLR3 and RIG-I to induce innate immune responses in other organisms (Goubau et al. 2014; Jensen and Thomsen 2012; Loo et al. 2008), but the observed response diverged markedly from the VSV infection, which is also sensed by RIG-I but nonetheless failed to induce an interferon alpha response.

As observed for Mx1, only Reo-infected cells showed a significant enrichment of differentially expressed genes involved in the type I IFN response (FDR-adjusted p-value=9.05×10⁻³; normalized enrichment score, NES=3.68). These genes contain the consensus transcription factor binding sites in the promoters that are mainly regulated by the transcription factor STAT1 and the interferon regulatory factors (IRF) family, such as IRF1, IRF3, IRF7 and IRF8 (FIG. 1E). These results are consistent with observations that the IRE family transcription factors activate downstream immune responses in virus-infected mammalian cells (Honda and Taniguchi 2006; Ivashkiv and Donlin 2014). In contrast, VSV and EMCV failed to trigger anti-viral related mechanisms (e.g., type I IFN responses) downstream of IFNβ (FIG. 1D). Examples of a few pathways that were stimulated included ‘immune system’ (including adaptive/innate immune system and cytokine signaling in immune system) in VSV (FDR-adjusted p-value=1.49×10⁻²; normalized enrichment score, NES=1.99) and the ‘G2M checkpoint’ in EMCV (p-value=8.95×10⁻³; NES=2.64). However, neither VSV nor EMCV infection activated known upstream activators (FIG. 1E) of type I IFN pathways when analyzed with Ingenuity Pathway Analysis (IPA) (Kramer et al. 2014).

### Poly I:C Induces a Robust Type I Interferon Response in CHO Cells

Type I IFN responses limit viral infection (Perry et al. 2005; Sadler and Williams 2008; Schoggins and Rice 2011; Taniguchi and Takaoka 2002), and innate immune modulators (Bohlson 2008; Mutwiri et al. 2007; Olive 2012) mimic pathogenic signals and stimulate pattern recognition receptors (PRRs), leading to the activation of downstream immune-related pathways. Intracellular PRRs, including toll-like receptors (TLR) 7, 8 and 9, and cytosolic receptors RIG-I or MDA5, can sense viral nucleic acids and trigger the production of type I IFN. This example sought to determine whether CHO cell viral resistance could be improved by innate immune modulators.

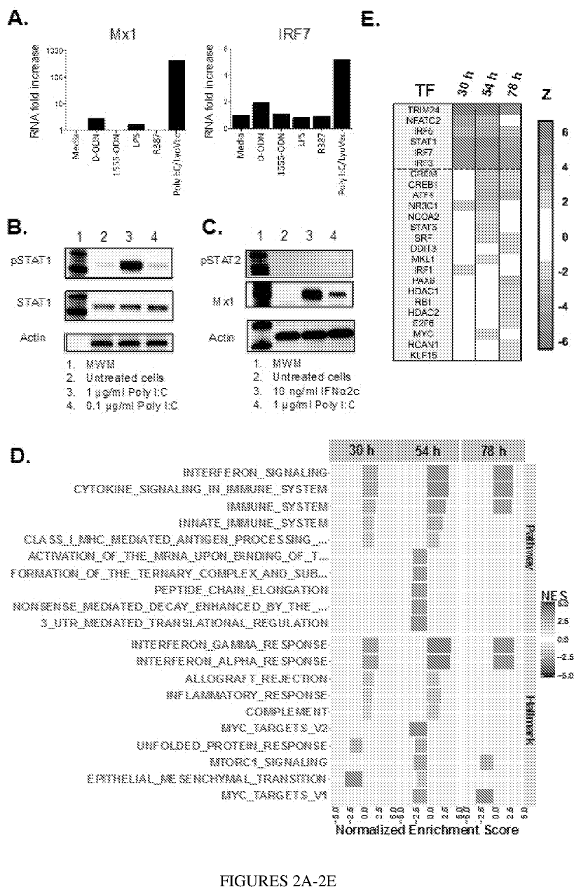

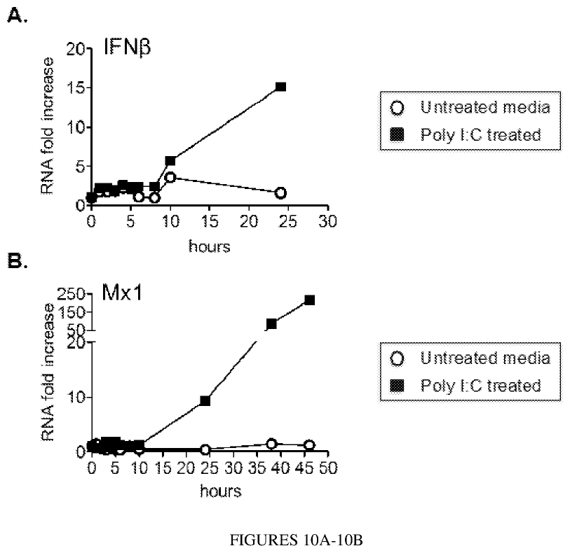

CHO PRRs have not been studied extensively, so the ability of synthetic ligands to stimulate their cognate receptors to induce a type I IFN response was first assessed. CHO cells were incubated with LPS (TLR4 ligand), CpG-oligodeoxynucleotide (ODN) type D (activates TLR9 on human cells), ODN-1555 (activates TLR9 on murine cells), imidazoquinoline R837 (TLR7/8 ligand) and poly I:C-Low molecular weight/LyoVec (poly I:C) (activates the RIG-I/MDA-5 pathway), and subsequently tested for changes in expression of IFN stimulated genes with anti-viral properties. After 24 h of culture, gene expression levels of IRF7 and Mx1 increased significantly in cells treated with poly I:C but not in those treated with any of the other innate immune modulators (FIG. 2A). Furthermore, STAT1 and STAT2 phosphorylation and Mx1 protein levels were elevated following treatment with poly I:C or exogenous interferon-alpha (IFNα), which was used as a control (FIGS. 2B and 2C). By monitoring changes in the gene expression levels of IFNβ and Mx1 in the cells, it was established that 16-20 h would be an adequate time interval for treating cells with poly I:C prior to infection (FIGS. 10A-10B).

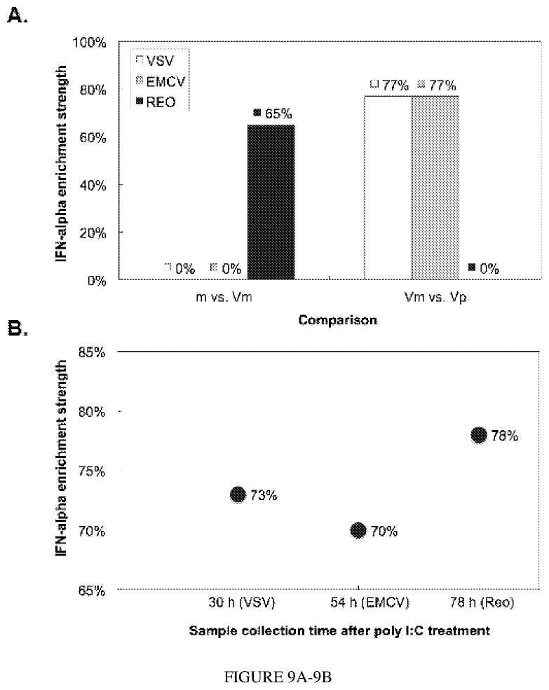

Next, the type I IFN response induced by poly I:C was characterized by analyzing the transcriptome of untreated vs. treated CHO cells. Cells were cultured with poly I:C in the media for 30, 54 and 78 h after an initial 16 h pre-incubation period. GSEA of the RNA-Seq data demonstrated that poly I:C induced a strong ‘innate immune response’ in comparison to untreated cultures (media) (in vs. p; (p-value, NES, Enrichment strength)=(8.08×10⁻³, 2.98, 73%), (1.57×10⁻², 3.95, 70%) and (3.91×10⁻³, 3.58, 78%)) evident at all the tested time points (FIGS. 2D and 9B). In addition, it activated several upstream regulators of the type I IFN pathways (FIG. 2E). It was noted that the strength of the gene set enrichment (see EXAMPLE 2) of the innate immune response induced by poly I:C (m vs. p) was stronger than the innate immune response seen for Reo infection alone (m vs. Vm in FIGS. 9A-9B). Thus, CHO cells can activate the type I IFN signaling (JAK-STAT) pathway in response to poly I:C and display an anti-viral gene signature, which was sustained for at least 4 days.

Poly I:C-Induced Type I Interferon Response Protect CHO Cells from RNA Virus Infections

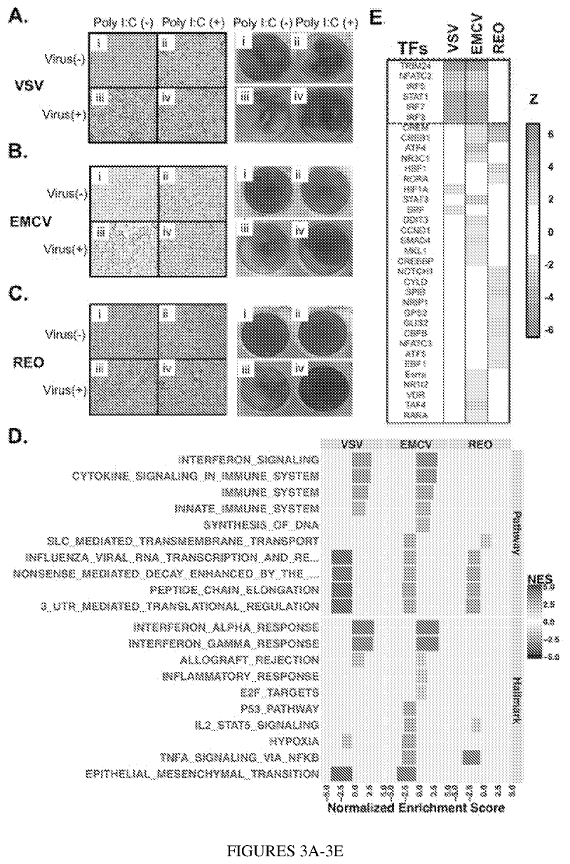

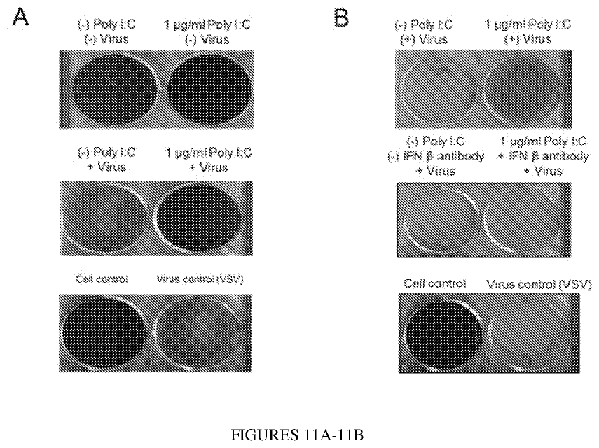

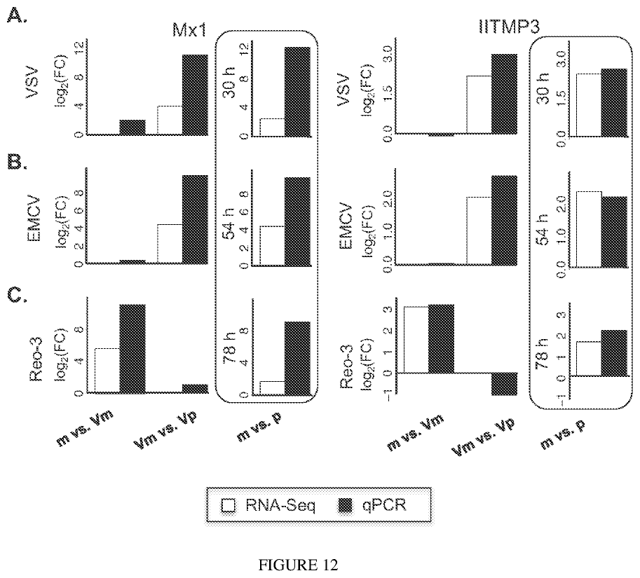

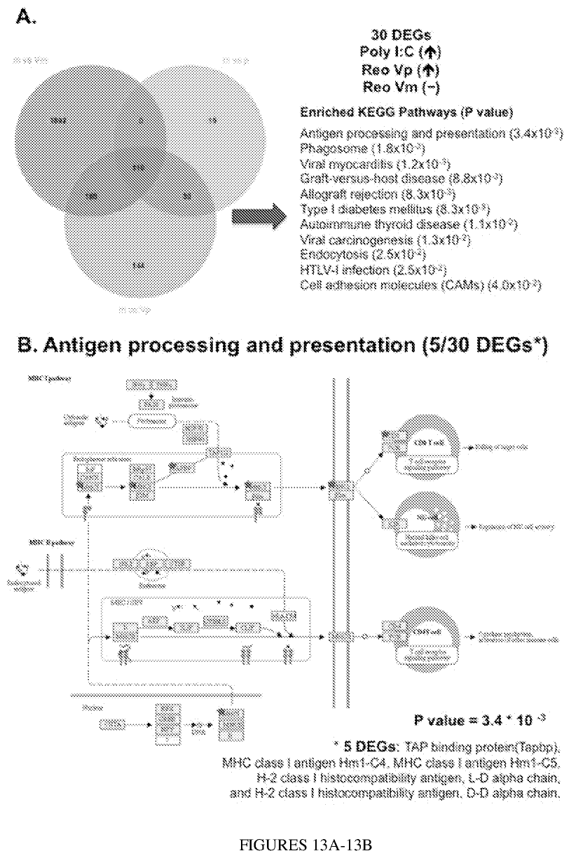

It was next examined if the type I IFN response, induced by poly I:C, could protect CHO cells from RNA virus infections. It was found that poly I:C pre-treatment protected CHO cells against viral infection through the IFNβ-mediated pathway (FIGS. 11A-11B), and that poly I:C protected against all three viruses tested (FIGS. 3A-C). Cell morphology differed notably between cultures infected with virus (Vm), control uninfected cells (m), and poly I:C pre-treated cultures (p and Vp) (FIGS. 3A-3C). These morphological changes correlated with the cytopathic effect observed in the cell monolayers (FIGS. 3A-3C, right panels). At 78 h, the extent of cell culture damage by Reo, however, was milder than by VSV and EMCV at a shorter incubation times (30 h and 54 h, respectively) (FIGS. 3A-3C), possibly since Reo induced higher levels of anti-viral related genes in the CHO cells but VSV and EMCV did not (FIGS. 1C, 1D and 1E). Notably, although poly I:C pre-treatment conferred protection of CHO cells to all three viral infections (FIG. 3A-3C), striking transcriptomic differences were observed. Poly I:C pre-treatment significantly activated immune-related pathways and up-regulated type I IFN-related gene expression in CHO cells infected with VSV and EMCV when compared to non-poly I:C pre-treated cells that were infected (Vm vs. Vp) (FIGS. 3D-3E). Poly I:C pre-treatment was sufficient to induce a protective type I IFN response to VSV and EMCV. For Reo infection, however, pre-treatment with poly I:C did not further increase the levels of expression of IFN associated genes over those observed in poly I:C-untreated, infected cells. The lack of enhanced expression of antiviral genes in Reo Vm vs. Vp observed in the GSEA was further confirmed by Taqman analysis. A similar level of expression of anti-viral Mx1 and IITMP3 genes (Diamond and Farzan 2013; Li et al. 2013; Pillai et al. 2016; Verhelst et al. 2013) was obtained for CHO cells independently infected with Reo (Vm) treated with poly I:C (p) or pre-treated with poly I:C and infected (Vp), which resulted in no differences in transcript levels when we compared Vm vs. Vp (FIG. S5C). Nevertheless, the outcome of infection was surprisingly different in Vm or Vp samples. To understand these differences, genes that were differently modulated by poly I:C treatment in the context of Reo infection were identified. Indeed, 30 genes (FIGS. 13A-13B) that were significantly up regulated (adjusted p-value<0.05, fold change>1.5) in the comparisons of m vs. Vp and m vs. p but not in the comparison of m vs. Vm. These genes are significantly enriched in 11 KEGG pathways related to host-immune response (e.g., antigen processing and presentation, p-value=3.4×10⁻³) and processes important to virus infection (e.g., endocytosis, p-value=2.5×10⁻²). It was also observed that many of these genes significantly enriched molecular functions: 1) RNA polymerase II transcription factor activity (11 genes; GO:0000981 FDR-adjusted p-value<1.30×10⁻¹⁵) and 2) nucleic acid binding transcription factor activity (12 genes GO:0001071 FDR-adjusted p-value<3.54×10⁻¹⁵) by gene set enrichment analysis (see EXAMPLE 2). This suggests that poly I:C treatment, 16 hours prior to virus infection, pre-disposes the cell to adopt an antiviral state and might restore the host transcription machinery subverted by Reo virus resulting in the protection of the CHO cells.

The results revealed other processes that are differentially activated or repressed between Vm and Vp (FIG. 3D). For example, the top down-regulated Reactome pathways in the virus-infected cells are protein translational related processes: ‘nonsense mediated decay enhanced by the exon junction complex’ (p-value=3.32×10⁻², NES=−3.50), ‘peptide chain elongation’ (p-value=3.32×10⁻², NES=−3.59), and ‘3′-UTR mediated translational regulation’ (p-value=3.38×10², NES=−3.61). These results agree with studies showing viral hijacking of the host protein translation machinery during infection (Walsh et al. 2013), and that the activation of interferon-stimulated genes restrain virus infections by inhibiting viral transcription and/or translation (Schoggins and Rice 2011). All these results suggest that poly I:C treatment provides the cell with an advantageous immune state that counteracts viral escape mechanisms and results in cell survival.

### A STAT1-Dependent Regulatory Network Governs Viral Resistance in CHO Cells.

GSEA revealed that several transcriptional regulators were activated or repressed during different viral infections and poly I:C-treated cells (FIGS. 1C-1E, 2E, and 3E). Among these, six were consistently and significantly activated across different virus and media conditions (highlighted in dash rectangles; FIGS. 1C, 2E and 3E). These included NFATC2, STAT1, IRF3, IRF5, and IRF7, which were all activated in poly I:C pretreatment of CHO cells (m vs. p and Vm vs. Vp). These transcription factors are involved in TLR-signaling (IRF3, IRF5, and IRF7; (Honda and Taniguchi 2006)) and JAK/STAT signaling (NFATC2, STAT1, and TRIM24). The TLR signaling pathway is a downstream mediator in virus recognition/response and in activating downstream type-I interferon immune responses (Arpaia and Barton 2011; Kawai and Akira 2009; Thompson and Locarnini 2007). Meanwhile, the JAK/STAT pathway contributes to the antiviral responses by up-regulating interferon simulated genes to rapidly kill virus within infected cells (Aaronson and Horvath 2002; Au-Yeung et al. 2013; Li and Watowich 2014). Importantly, one mechanism by which STAT1 expression and activity may be enhanced is via the poly I:C induced repression of TRIM24, which inhibits STAT1. The crosstalk between TLR- and JAK/STAT-signaling pathways plays essential roles in virus clearance of the virus infected host cells (Hu and Ivashkiv 2009).

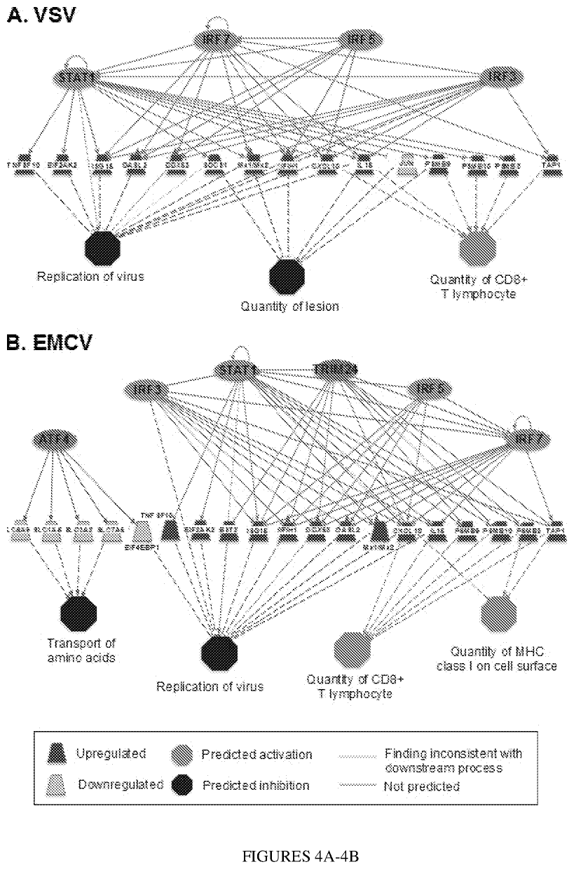

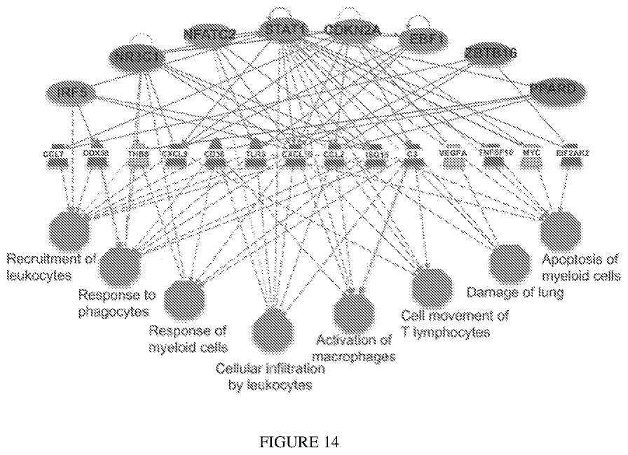

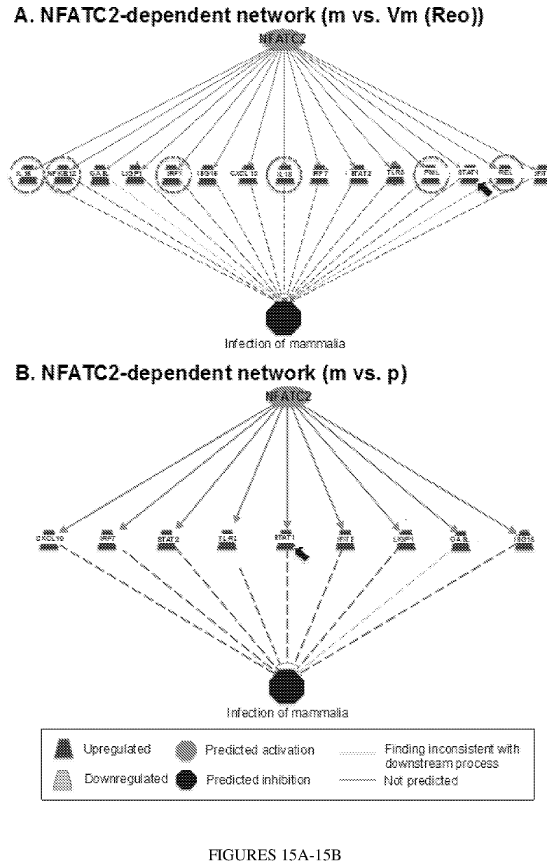

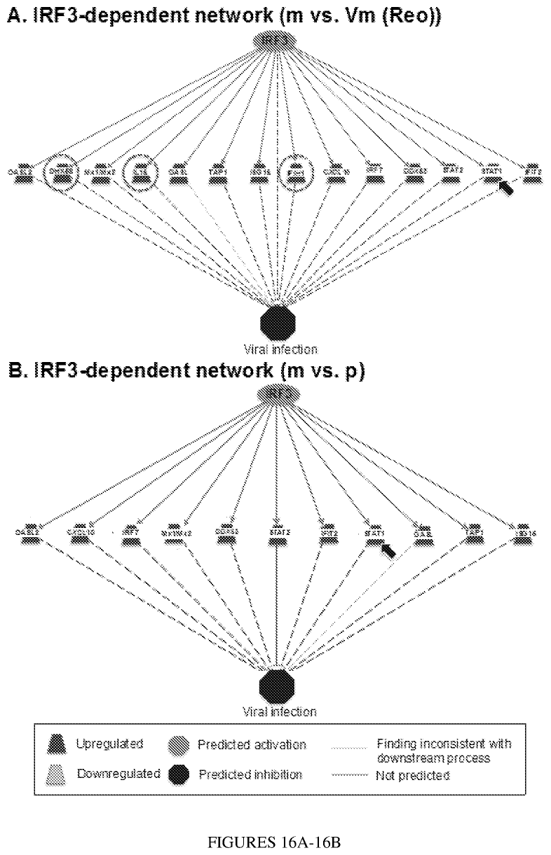

The roles of upstream regulators were further investigated by examining the expression of their downstream target genes. Table 2 shows the results of the regulatory pathways emanating from poly I:C treatment and their expected effects on the downstream phenotypes. Regulatory networks were identified that capture the anti-viral response of the cells (FIGS. 4A and 4B for VSV and EMCV respectively). The networks are predominantly regulated by these same 6 transcription factors (NFATC2, STAT1, IRF3, IRF5, IRF7, and TRIM24), which can regulate many genes that together inhibit VSV and EMCV virus replication in poly I:C pretreated cells (Table 2). The activation of this STAT1-dependent regulatory network by poly I:C-treated media leads to the induction of several immune-related responses (e.g., recruitment for leukocytes; FIG. 14). The induction of the STAT1-dependent regulatory network with poly I:C pretreatment, and the subsequent viral resistance suggests that the network may have protective power against virus infection. While the STAT1-dependent regulatory network did not apparently emerge when comparing the poly I:C pre-treatment compared to the untreated Reo infected cells (Vm vs. Vp), because those pathways are natively activated by Reo since poly I:C is a structural analog of double-stranded RNA (Fortier et al. 2004). For example, NFATC2-dependent network (FIGS. 15A-15B) and IRF3-dependent network (FIGS. 16A-16B) are two example networks that presented in both of the comparisons m vs. p and m vs. Vm.

### Deletion of Trim24 and Gfi1 Induced CHO Cell Innate Immunity and Viral Resistance

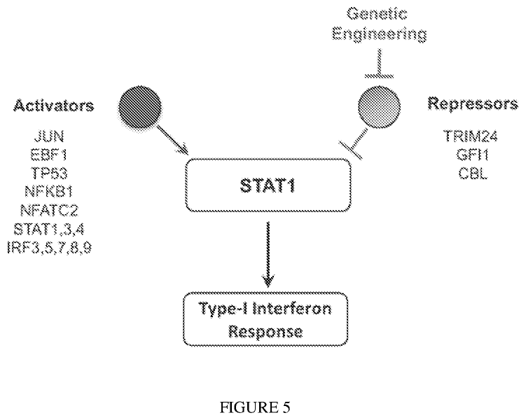

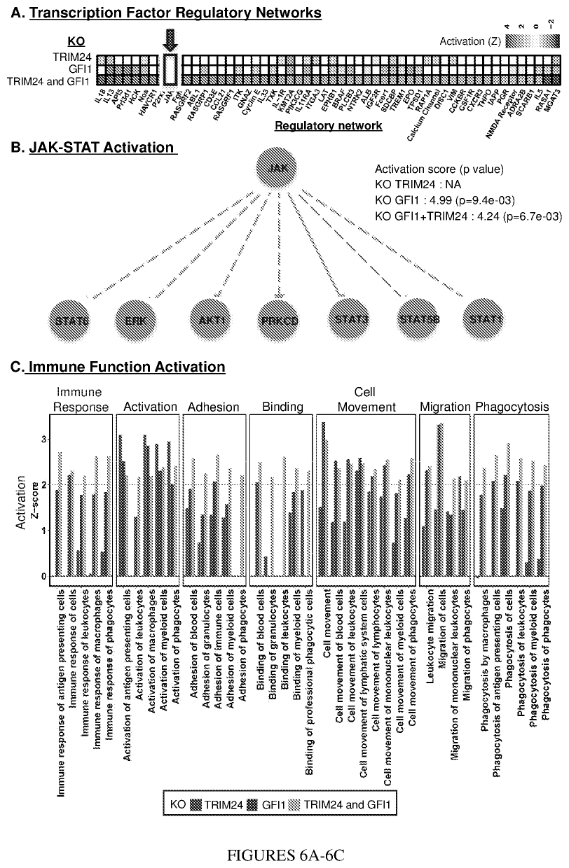

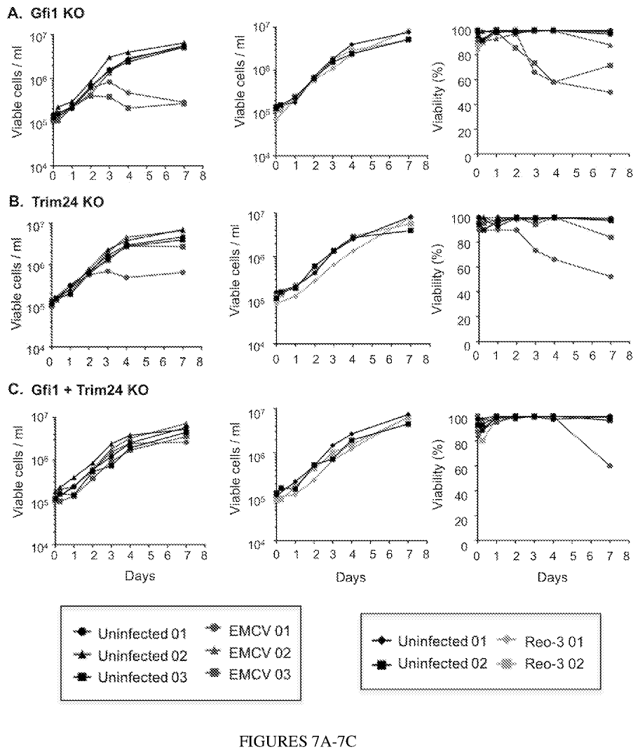

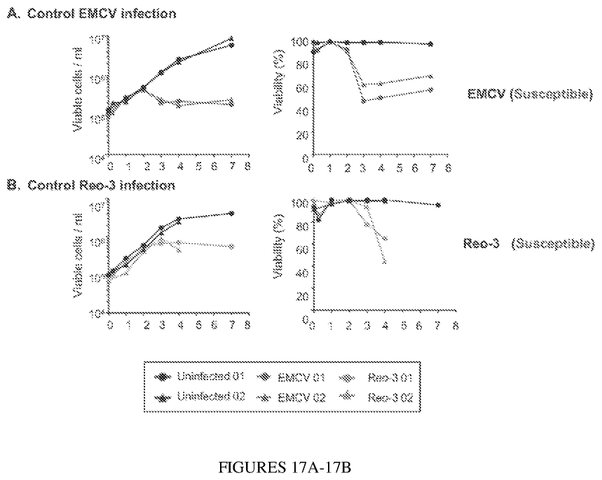

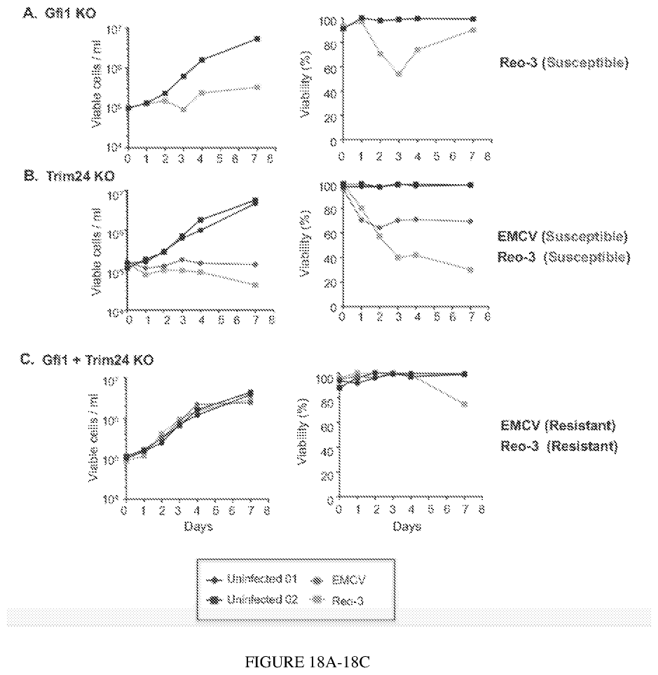

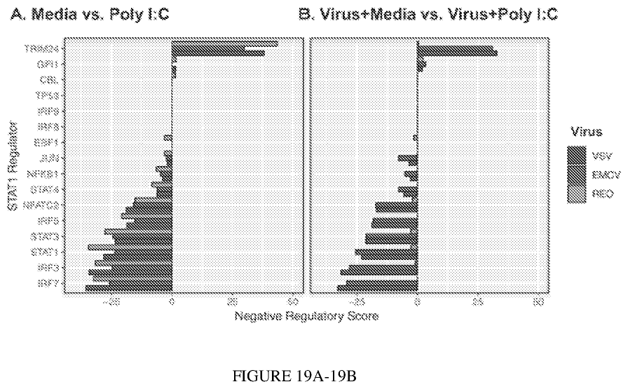

With the STAT1 network potentially contributing to viral resistance, upstream regulators were sought that could be modulated to naturally induce STAT1. That identified sixteen statistically significant (p<0.05) upstream regulators, including 13 positive and 3 negative regulators of Stat1 using IPA (FIG. 5). It was hypothesized that the deletion of the most active repressors of Stat1 could improve virus resistance by inducing Stat1 expression and the downstream type I IFN antiviral response (FIG. 5). Three Stat1 repressors (Trim24, Gfi1 and Cb1) with a negative regulatory score were identified and therefore having potential for inhibiting Stat1 based on the RNA-Seq differential expression data (see details in FIGS. 19A-19B). However, Cb1 did not present in samples involving Reo virus infection. Therefore, the two negative regulators, Gfi1 (Sharif-Askari et al. 2010) and Trim24 (Tisserand et al. 2011), of Stat1 were selected as targets for genetic engineering (FIG. 5 and Table 4) and subsequently tested their susceptibility to Reo and EMCV. To evaluate the impact of gene editing on the engineered CHO-S cells, RNA-Seq was conducted in uninfected single (Gfi1 or Trim 24) or double (Gfi1+Trim 24) KO cell lines (FIGS. 6A-6C). The results revealed that these cells had increased transcript levels of a number of genes involved in innate immunity pathways, such as those mediated by interleukins (ILs) (e.g. IL-33 pathway (IL-1R, IL-5, IL-13, IL-33) and IL-18) (FIG. 6A) and STAT (e.g., STAT1, 3, 5B and 6)-related genes (FIG. 6B), leading to the upregulation of several immune functions (FIG. 6C) that could limit virus infection. Subsequently and as a proof of concept, the virus susceptibility of the cells was evaluated using Reo-3 and EMCV. It was found that the that the Trim24 and Gfi1 single knockout clones (FIG. 7A-7C) show resistance to Reo but moderate or no resistance against EMCV, compared to positive controls (FIGS. 17A-17B). However, the Gfi1 and Trim24 double knockout (FIG. 7C) showed resistance to both viruses tested, even when cultured with virus for a second week (FIGS. 18A-18B). Together these results show that the regulatory network contributes to antiviral mechanisms of CHO cells, which could possibly be harnessed to obtain virus resistant CHO bioprocesses.

These results suggest that the genomes of these RNA viruses are sensed by the same RIG-I/TLR3 receptors of the host cell, even if these RNA viruses of different families have found mechanisms to overcome the innate immune mechanisms of the CHO cells (FIG. 1). Activation of RIG-I/TLR3 with the ligand Poly I:C prior to virus infection gives an advantage to the host cell over the virus by inducing a robust type I IFN response allowing its survival. A similar outcome appears to be reached by deleting two of the type I IFN pathway negative regulators. The systems biology approach to identifying transcription factors impacting RNA virus infection can be replicated in the future for other virus classes, such as DNA viruses (e.g. MVM) which use other mechanisms for viral sensing such as TLR9, which is not expressed in CHO cells, therefore making CHO susceptible to MVM infection. Thus, using the present invention approach, regulators of innate immunity can be provided to make DNA virus resistance cells by simulating TLR9 or its downstream activities in CHO cells with the use of CpG ODN to induce a TLR9-driven type I IFN response on the cell.

### Example 2

### Gene Set Enrichment Analysis (GSEA) and Upstream Regulator (Transcriptional Factor) Analysis

### Gene Set Enrichment Analysis (GSEA) and Enrichment Strength Analysis

GSEA was performed using the Broad Institute GSEA software (Subramanian et al. 2005). A ranked list of genes (adjusted p-values<0.05) was made using the differential expression values (Fold change in the log₂ scale) from differential gene expression analysis were run through the GSEA pre-ranked protocol. GSEA-pre-rank analysis was processed to detect significant molecular signature terms (‘Hallmark’ (50) and ‘Reactome’ (674) gene sets from the MSigDB were used here) for the differential expressed genes. Note that, the criteria for considering a molecular signature term as significant are: 1) after Benjamini-Hochberg false discovery correction, molecular signature terms with adjusted p-values less than 0.05; and 2) there are >30 genes presented in the gene list of this molecular signature terms.

The leading edge analysis allows for the GSEA to determine which subsets (referred to as the leading edge subset) of genes contributed the most to the enrichment signal of a given gene set's leading edge or core enrichment (Subramanian et al. 2005). The leading edge analysis is determined from the enrichment score (ES), which is defined as the maximum deviation from zero. The enrichment strength describes the strength of the leading-edge subset of a gene set (i.e., the interferon-alpha response in this study) (Subramanian et al. 2005). Specifically, if the gene set is entirely within the first N positions in the ranked differentially expressed gene list, then the signal strength is maximal or 100%. If the gene set is spread throughout the list, then the signal strength decreases towards 0%.

### Upstream Regulator (Transcriptional Factor) Analysis

The upstream regulators were predicted using the Ingenuity IPA Upstream Regulator Analysis Tool by calculating a regulation Z-score and an overlap p-value (Kramer et al. 2014), which were based on the number of known target genes of interest pathway/function, expression changes of these target genes and their agreement with literature findings. It was considered significantly activated (or inhibited) with an overlap p-value less than 0.05 and an IPA activation |Z-score|≥1.96. Note that, the criteria for generating the resulting table (Table 2) from IPA are: 1) Total nodes>=10, and 2) Consistency score>=5.00. Consistency score is an IPA measurement (Kramer et al. 2014) for measuring the consistency of a predicted network (capturing regulator-target-function relationships) from RNA-Seq data with literature knowledge. The higher consistency scores of the predicted regulatory networks denote better consistency with literature support than the predicted regulatory networks with lower consistency scores.

Type I IFN Protects CHO Cells from VSV Infection

CHO cells failed to make a significant IFN response when infected with virus. It is well documented that type I IFN response is necessary to limit the extent of viral infection both in a cell culture and in vivo. Thus, this analysis sought to determine if the susceptibility to the virus was due to unresponsiveness of the cells to IFN rather than lack of ability to generate such a response. In order to simplify the screening, we first concentrated on VSV. Cells were seeded in 96-well plates and treated with human or murine type I IFN protein preparations for 24 h, prior to the addition of serially diluted VSV (1:10) (FIG. 8). Infection progressed for 24 h and cultures were stained with crystal violet (CV) to assess the extent of the protection by cytopathic effect. All IFN preparations limited viral cytopathic effect (FIG. 8). Of note, human IFNβ had the most potent anti-VSV effect of all the interferons tested, at least at the dose used in the experiment (FIG. 8). These results indicate that CHO cells have a functional IFNα/β receptor and that its activation confers resistance of CHO cells to VSV infection.

### Poly I:C Pre-Treatment of CHO Cells Protects Against Viral Infection Through the IFNβ-Mediated Pathway.

It was next examined if the type I IFN response induced by poly I:C could protect CHO cells from RNA virus infections by evaluating effect of poly I:C on CHO susceptibility to VSV infection. Cells were cultured with 1 μg/ml of poly I:C for 24 h prior to infection with VSV (MOI of 0.1). As in previous experiments, the control poly I:C-treated CHO cell monolayer remained intact during the length of the experiment (48 h) indicating that poly I:C per se was not toxic for the cells (FIG. 11A). In contrast, disruption of the CHO cell monolayer was evident in wells where VSV was added, but not in wells where CHO cells were pre-incubated with poly I:C (FIGS. 11A and 11B). Moreover, the poly I:C-induced anti-viral response of the cell was IFNβ-dependent, as demonstrated by addition of a neutralizing antibody to IFN-β (FIG. 11B). These results suggest that poly I:C treatment provides the cell with an advantageous immune state by activating the IFNβ-mediated pathway that counteracts viral escape mechanisms and results in cell survival.

### Identification of the STAT1 Upstream Regulators.

The upstream regulators of STAT1 were identified by the three steps. First, collect all the upstream regulators predicted using the Ingenuity IPA Upstream Regulator Analysis Tool in the RNA-Seq data of the comparisons: m vs. p (media vs poly I:C treated media) and Vm vs. Vp (virus+media vs virus+poly I:C treated media)). Second, further select those IPA predicted upstream regulators that can regulate STAT1 gene with literature evidences (Table 4). Third, define the negative regulatory score as below.

$$Negative regulatory score = - log 10 ( P - value ) \times Regulatoion Direction$$

$$Regulation Direction = \{ \begin{matrix}1 ( Repressor ) \\ 0 ( Unknown ) \\ - 1 ( Activator )\end{matrix}$$

The p-value (Table 4) here is calculated using Fisher's Exact Test for measuring whether there is a statistically significant overlap between the differentially expressed genes in our dataset genes and the genes that are regulated by a TF, as reported in IPA. The higher negative regulatory score of a TF represents the larger potential in inhibiting STAT1 based on the RNA-Seq differential expression data (FIGS. 19A-19B).

### Tables

TABLE 1
Study prototype viruses and MOI on CHO-K1 cells.

| | | Genomic | Referenced | |
|---|---|---|---|---|
| | | nucleic | CHO cell | |
| | Virus | acid | culture | |
| Virus | family | nature | infection | MOI |
| Vesicular | Rabdoviridae | ss (−) | Potts, 2008 | 0.003 |
| stomatitis | | RNA | | |
| virus (VSV) | | | | |
| Encephalomyocar- | Picornaviridae | ss (+) | Potts, 2008 | 0.007 |
| ditis virus | | RNA | | |
| (EMCV) | | | | |
| Reovirus 3 (Reo) | Reoviridae | ds RNA | Wisher, | 0.0013 |
| | | | 2005; | |
| | | | Rabenau | |
| | | | 1993 | |

TABLE 2A
The downstream effects of the upstream regulators from the comparison of m vs. p.

| | | Total | | Target | | | |
|---|---|---|---|---|---|---|---|
| | Consistency | nodes (TF, | | gene*^{b} | Biological | | |
| Virus | score | TG, BP) | TF*^{a} | (TG) | Process*^{c} | Relations*^{d} | |
| VSV | 5.82 | 21 | STAT1, | CASP1, | Inhibit | 6/15 | (40%) |
| | | (5, 13, | IRF3, IRF5, IRF7, | CXCL10, | Replication | | |
| | | 3) | NFATC2 | DDX58, | of virus. | | |
| | | | | EIF2AK2, | | | |
| | | | | IFIH1, | | | |
| | | | | IL15, | Activate | | |
| | | | | ISG15, | Activation of | | |
| | | | | Mx1/Mx2, | phagocytes; | | |
| | | | | OASL2, | Apoptosis of | | |
| | | | | PELI1, | antigen | | |
| | | | | PML, | presenting | | |
| | | | | SOCS1, | cells. | | |
| | | | | TNFSF10 | | | |
| EMCV | 22.47 | 48 | STAT1, | BST2, C3, | Inhibit | 21/84 | (25%) |
| | | (7.29.12) | IRF3, IRF5, IRF7, | CASP1, | Replication | | |
| | | | NFATC2, TRIM24, | CXCL10, | of virus; | | |
| | | | NCOA2 | DDX58, | Infection by | | |
| | | | | EGR2, | RNA virus; | | |
| | | | | EIF2AK2, | Infection of | | |
| | | | | GBP2, | central nervous | | |
| | | | | IHH1, | system. | | |
| | | | | IFIT1B, | Activate | | |
| | | | | IFIT2, | Antiviral | | |
| | | | | IFITM3 | response; | | |
| | | | | (IITMP3), | Clearance of | | |
| | | | | Igtp, IL15, | virus; | | |
| | | | | ISG15, | Immune | | |
| | | | | Mx1/Mx2, | response of | | |
| | | | | MYC, | antigen | | |
| | | | | OASL2, | presenting | | |
| | | | | PML, | cells; | | |
| | | | | PSMB10, | Immune | | |
| | | | | PSMB8, | response of | | |
| | | | | PSME2, | phagocytes; | | |
| | | | | PTGS2, | Cytotoxicity | | |
| | | | | SPP1, | of leukocytes; | | |
| | | | | STAT2, | Function of | | |
| | | | | TAPI, | leukocytes; | | |
| | | | | TLR3, | Infiltration | | |
| | | | | TNFSF10, | by T | | |
| | | | | TRAFD1 | lymphocytes; | | |
| | | | | | Quantity of | | |
| | | | | | MHC Class I | | |
| | | | | | of cell | | |
| | | | | | surface; Cell | | |
| | | | | | death of | | |
| | | | | | myeloid cells. | | |
| REO | 27.80 | 30 | STAT1, | C3, | Activate | 11/64 | (17%) |
| | | (8, 14, | IRF5, NFATC2, | CCL2, | Activation of | | |
| | | 8) | NR3C1, PPARD, ZBTB16, | CCL7, | macrophages; | | |
| | | | CDKN2A, EBF1 | CD36, | Apoptosis of | | |
| | | | | CXCL10, | myeloid | | |
| | | | | CXCL9, | cells; Cell | | |
| | | | | DDX58, | movement of | | |
| | | | | EIF2AK2, | T lymphocytes; | | |
| | | | | ISG15, | Cellular | | |
| | | | | MYC, | infiltration by | | |
| | | | | THBS1, | leukocytes; | | |
| | | | | TLR3, | Damage of | | |
| | | | | TNFSF10, | lung; | | |
| | | | | VEGFA | Recruitment of | | |
| | | | | | leukocytes; | | |
| | | | | | Response of | | |
| | | | | | myeloid | | |
| | | | | | cells; | | |
| | | | | | Response of | | |
| | | | | | phagocytes. | | |
| REO | 7.56 | 12 | CDKN2A, ZBTB16 | C3, | Activate | 1/6 | (17%) |
| | | (2, 7, | | CCL2, | Cell | | |
| | | 3) | | CCL7, | movement of | | |
| | | | | CXCL10, | T lymphocytes; | | |
| | | | | CXCL9, | Recruitment of | | |
| | | | | MYC, | leukocytes; | | |
| | | | | VEGFA | Survival of | | |
| | | | | | organism. | | |

*^{a,b}The upstream regulators (STAT1 is highlighted in bold face) and the antiviral relating genes.
*^{c}The biological functions known to associated with the regulatory networks annotated by the IPA.
*^{d}The number of identified relationships and the total relationships that represent the known regulatory relationships between regulators and functions supported by literatures annotated by the IPA.

TABLE 2B
The downstream effects of the upstream regulators from the comparison of Vm vs. Vp.

| | | Total | | | | |
|---|---|---|---|---|---|---|
| | | nodes | | | | |
| | Consistency | (TF, TG, | | Target genes | Biological Process | |
| Virus | score | BP) | TF | (TG) | (BP) | Relations |
| VSV | 8.00 | 22 | STAT1, | CXCL10, DDX58, | Inhibit | 2/12 (17%) |
| | | (4, 15, | IRF3, IRF5, | EIF2AK2, IFIH1, | Replication of | |
| | | 3) | IRF7 | IL15, ISG15, JUN, | virus; Quantity of | |
| | | | | Mx1/Mx2, OASL2, | lesion. | |
| | | | | PSMB10, PSMB8, | Activate | |
| | | | | PSMB9, SOCS1, | Quantity of CD8+ | |
| | | | | TAP1, TNFSF10 | T lymphocyte. | |
| EMCV | 12.16 | 29 | STAT1, | BST2, CXCL10, | Inhibit | 3/24 (13%) |
| | | (6, 19, | IRF3, IRF5, | DDX58, EIF2AK2, | Replication of | |
| | | 4) | IRF7, TRIM24, | EIF4EBP1, IFIH1, | virus; Transport of | |
| | | | ATF4 | IL15, ISG15, | amino acids. | |
| | | | | Mx1/Mx2, OASL2, | Activate | |
| | | | | PSMB10, PSMB8, | Quantity of CD8+ | |
| | | | | PSMB9, SLC1A5, | T lymphocyte; | |
| | | | | SLC3A2, SLC6A9, | Quantity of MHC | |
| | | | | SLC7A5, TAP1, | Class I on cell | |
| | | | | TNFSF10 | surface. | |
| EMCV | 7.91 | 18 | CCND1, | AREG, CCND2, | Inhibit | 7/12 (58%) |
| | | (2, 10, | SMAD4 | EREG, GJA1, | Arthritis; Cell cycle | |
| | | 6) | | HSPA8, ITGAV, | progression; Cell | |
| | | | | NFKBIA, PTGS2, | viability; Growth of | |
| | | | | SOX4, SPP1 | ovarian follicle; | |
| | | | | | Proliferation of | |
| | | | | | cells. | |
| | | | | | Activate | |
| | | | | | Edema. | |
| EMCV | 6.96 | 19 | MKL1, | CAMP, CCL2, HLA-A, | Inhibit | 7/14 (50%) |
| | | (2, 10, | VDR | ICAM1, IL6, | Cancer; Quantity of | |
| | | 7) | | MMP9, PTGS2, | interleukin; | |
| | | | | RELB, SPP1, TNC | Rheumatic Disease; | |
| | | | | | Development of | |
| | | | | | body trunk. | |
| | | | | | Activate | |
| | | | | | Cell death of | |
| | | | | | connective tissue | |
| | | | | | cells; Nephritis; | |
| | | | | | Organismal death. | |
| REO | 5.61 | 21 | GFI1, NR1H3, | ACACB, CAV1, | Inhibit | 1/12 (8%) |
| | | (4, 14, | NRIP1, | CD36, CSF3, ETS1, | Oxidation of | |
| | | 3) | PPARG | ID2, IL6, LDLR, | carbohydrate; | |
| | | | | LPL, NFKBIA, | Production of | |
| | | | | PDK2, PDK4, | leukocytes; | |
| | | | | PPARA, SLC2A1 | Quantity of vldl | |
| | | | | | triglyceride in | |
| | | | | | blood. | |

TABLE 3
Statistics of differentially expressed genes.

| | Differential | VSV | | EMCV | | REO | |
|---|---|---|---|---|---|---|---|
| Comp. | expression* | Down | Up | Down | Up | Down | Up |
| 1 | m vs. Vm | 1 | 24 | 8 | 16 | 1688 | 1945 |
| 2 | m vs. p | 58 | 245 | 269 | 422 | 28 | 136 |
| 3 | Vm vs. Vp | 271 | 281 | 275 | 337 | 1859 | 1657 |

a. *m: untreated − uninfected (media control); p: poly I:C treated − uninfected; Vm: untreated − virus infected; Vp: poly I:C treated − virus infected.
(Note that the criteria for identifying DEGs were: adjusted p-value < 0.05, and |Fold Change| > 1.5.)

TABLE 4
Upstream regulators of STAT1 predicted by IPA.

| | m vs p | | | Vm vs Vp | | | Regulation | |
|---|---|---|---|---|---|---|---|---|
| TF | VSV | EMCV | REO | VSV | EMCV | REO | Direction | Reference |
| TRIM24 | 8.90E−39 | 1.46E−30 | 2.65E−44 | 1.52E−33 | 1.12E−31 | 3.78E−01 | Inhibited | 21768647 |
| IRF7 | 2.55E−36 | 1.35E−26 | 2.99E−33 | 8.99E−34 | 6.51E−30 | | Activate | 23300459 |
| IRF3 | 5.13E−35 | 1.42E−25 | 2.02E−32 | 2.46E−32 | 8.35E−29 | 9.35E−02 | Activate | 23300459 |
| STAT1 | 6.52E−29 | 2.25E−24 | 2.63E−35 | 6.20E−24 | 2.45E−26 | 1.76E−03 | Activate | 22171011; |
| | | | | | | | | 24412616 |
| STAT3 | 4.10E−24 | 4.03E−25 | 2.08E−28 | 4.36E−22 | 5.49E−22 | 1.48E−03 | Activate | 12060750; |
| | | | | | | | | 12060750 |
| NFATC2 | 1.07E−19 | 1.38E−16 | 4.64E−16 | 1.27E−17 | 6.30E−18 | 7.53E−03 | Activate | 22078882 |
| IRF5 | 2.44E−19 | 3.39E−16 | 1.56E−21 | 1.15E−19 | 4.94E−19 | | Activate | 23300459 |
| STAT4 | 6.16E−07 | 7.28E−07 | 5.15E−09 | 2.18E−06 | 1.71E−08 | | Activate | 22968462 |
| IRF9 | 1.17E−05 | 4.09E−04 | 2.78E−06 | 7.80E−05 | 2.48E−04 | | Unknown | 20089923 |
| IRF8 | 1.00E−04 | 1.41E−04 | 2.94E−06 | 2.40E−04 | 6.53E−06 | 3.65E−02 | Unknown | 22805310 |
| NFKB1 | 1.00E−04 | 1.66E−05 | 3.52E−07 | 1.31E−03 | 5.28E−06 | | Activate | 14568969 |
| TP53 | 1.05E−02 | 7.88E−04 | 1.13E−03 | 7.81E−04 | 6.44E−05 | 7.20E−02 | Unknown | 16611991 |
| JUN | 1.22E−02 | 5.52E−03 | 8.04E−04 | 3.80E−04 | 1.51E−08 | | Activate | 20436908 |
| GFI1 | 3.45E−02 | | 2.24E−02 | 1.38E−02 | 3.65E−04 | 5.97E−03 | Inhibited | 20547752 |
| EBF1 | | | 7.76E−04 | | | 2.52E−02 | Activate | 24174531 |
| CBL | | | 4.27E−02 | | | | Inhibited | 11704862 |

Note that, the number in each virus column denote p-value of the enrichment (hypergeometric) of the differentially expressed TF target genes in that TF.

### REFERENCES

1. Aaronson D S, Horvath C M. 2002. A road map for those who don't know JAK-STAT. Science 296(5573):1653-5.
2. Ahmed M, McKenzie M O, Puckett S, Hojnacki M, Poliquin L, Lyles D S. 2003. Ability of the matrix protein of vesicular stomatitis virus to suppress beta interferon gene expression is genetically correlated with the inhibition of host RNA and protein synthesis. Journal Of Virology 77(8):4646-4657.
3. Anders S, Huber W. 2010. Differential expression analysis for sequence count data. Genome Biol 11(10):R106.
4. Anders S, Pyl P T, Huber W. 2015. HTSeq—a Python framework to work with high-throughput sequencing data. Bioinformatics 31(2):166-9.
5. Arpaia N, Barton G M. 2011. Toll-like receptors: key players in antiviral immunity. Curr Opin Virol 1(6):447-54.
6. Au-Yeung N, Mandhana R, Horvath C M. 2013. Transcriptional regulation by STAT1 and STAT2 in the interferon JAK-STAT pathway. JAKSTAT 2(3):e23931.
7. Berting A, Farcet M R, Kreil T R. 2010. Virus susceptibility of Chinese hamster ovary (CHO) cells and detection of viral contaminations by adventitious agent testing. Biotechnol Bioeng 106(4):598-607.
8. Bethencourt V. 2009. Virus stalls Genzyme plant. Nature Biotechnology 27:681.
9. Bohlson S S. 2008. Modulators of the innate immune response. Curr Drug Targets 9(2): 101.
10. Bolger A M, Lohse M, Usadel B. 2014. Trimmomatic: a flexible trimmer for Illumina sequence data. Bioinformatics 30(15):2114-20.
11. Chen C, Le H, Goudar C T. 2017. Evaluation of two public genome references for Chinese hamster ovary cells in the context of RNA-seq based gene expression analysis. Biotechnol Bioeng.
12. Diamond M S, Farzan M. 2013. The broad-spectrum antiviral functions of IFIT and IFITM proteins. Nat Rev Immunol 13(1):46-57.
13. Dinowitz M, Lie Y S, Low M A, Lazar R, Fautz C, Potts B, Sernatinger J, Anderson K. 1992. Recent studies on retrovirus-like particles in Chinese hamster ovary cells. Dev Biol Stand 76:201-7.
14. Dobin A, Davis C A, Schlesinger F, Drenkow J, Zaleski C, Jha S, Batut P, Chaisson M, Gingeras T R. 2013. STAR: ultrafast universal RNA-seq aligner. Bioinformatics 29(1):15-21.
15. FDA. 1998. Guidance for industry: Q5A viral safety evaluation of biotechnol-ogy products derived from cell lines of human or animal origin.
16. FDA. 2006. Guidance for industry: Characterization and qualification of cell substrates and other biological starting materials used in the produc-tion of viral vaccines for the prevention and treatment of infectious diseases.
17. Fomina-Yadlin D, Mujacic M, Maggiora K, Quesnell G, Saleem R, McGrew J T. 2015. Transcriptome analysis of a CHO cell line expressing a recombinant therapeutic protein treated with inducers of protein expression. J Biotechnol 212:106-15.
18. Fortier M E, Kent S, Ashdown H, Poole S, Boksa P, Luheshi G N. 2004. The viral mimic, polyinosinic:polycytidylic acid, induces fever in rats via an interleukin-1-dependent mechanism. Am J Physiol Regul Integr Comp Physiol 287(4):R759-66.
19. Garnick R L. 1998. Raw materials as a source of contamination in large-scale cell culture. Dev Biol Stand 93:21-9.
20. Goubau D, Schlee M, Deddouche S, Pruijssers A J, Zillinger T, Goldeck M, Schuberth C, Van der Veen A G, Fujimura T, Rehwinkel J and others. 2014. Antiviral immunity via RIG-I-mediated recognition of RNA bearing 5′-diphosphates. Nature 514(7522):372-+.
21. Green T J, Montagnani C. 2013. Poly I:C induces a protective antiviral immune response in the Pacific oyster (Crassostrea gigas) against subsequent challenge with Ostreid herpesvirus (OsHV-1 muvar). Fish Shellfish Immunol 35(2):382-8.
22. Gray L M, Lee J S, Gerling S, Kallehauge T, H Hansen A, Kol S, Lee G M, Pedersen L, Kildegaard H. One-step generation of triple knockout CHO cell lines using CRISPR Cas9 and fluorescent enrichment. Biotechnol J. 2015; 10:1446-56.
23. Haines K M, Vande Burgt N H, Francica J R, Kaletsky R L, Bates P. 2012. Chinese hamster ovary cell lines selected for resistance to ebolavirus glycoprotein mediated infection are defective for NPC1 expression. Virology 432(1):20-8.
24. Honda K, Taniguchi T. 2006. IRFs: master regulators of signalling by Toll-like receptors and cytosolic pattern-recognition receptors. Nat Rev Immunol 6(9):644-58.
25. Hsu H H, Araki M, Mochizuki M, Hori Y, Murata M, Kahar P, Yoshida T, Hasunuma T, Kondo A. 2017. A Systematic Approach to Time-series Metabolite Profiling and RNA-seq Analysis of Chinese Hamster Ovary Cell Culture. Sci Rep 7:43518.
26. Hu X, Ivashkiv L B. 2009. Cross-regulation of signaling pathways by interferon-gamma: implications for immune responses and autoimmune diseases Immunity 31(4):539-50.
27. Ivashkiv L B, Donlin L T. 2014. Regulation of type I interferon responses. Nature Reviews Immunology 14(1):36-49.
28. Jensen S, Thomsen A R. 2012. Sensing of RNA viruses: a review of innate immune receptors involved in recognizing RNA virus invasion. J Virol 86(6):2900-10.
29. Katze M G, He Y, Gale M, Jr. 2002. Viruses and interferon: a fight for supremacy. Nat Rev Immunol 2(9):675-87.
30. Kawai T, Akira S. 2009. The roles of TLRs, RLRs and NLRs in pathogen recognition. Int Immunol 21(4):317-37.
31. Kramer A, Green J, Pollard J, Jr., Tugendreich S. 2014. Causal analysis approaches in Ingenuity Pathway Analysis. Bioinformatics 30(4):523-30.
32. Lewis N E, Liu X, Li Y X, Nagarajan H, Yerganian G, O'Brien E, Bordbar A, Roth A M, Rosenbloom J, Bian C and others. 2013. Genomic landscapes of Chinese hamster ovary cell lines as revealed by the Cricetulus griseus draft genome. Nature Biotechnology 31(8):759-+.
33. Li H S, Watowich S S. 2014. Innate immune regulation by STAT-mediated transcriptional mechanisms. Immunol Rev 261(1):84-101.
34. Li K, Markosyan R M, Zheng Y M, Golfetto O, Bungart B, Li M, Ding S, He Y, Liang C, Lee J C and others. 2013. IFITM proteins restrict viral membrane hemifusion. PLoS Pathog 9(1):e1003124.
35. Loo Y M, Fornek J, Crochet N, Bajwa G, Perwitasari O, Martinez-Sobrido L, Akira S, Gill M A, Garcia-Sastre A, Katze M G and others. 2008. Distinct RIG-I and MDA5 signaling by RNA viruses in innate immunity. Journal Of Virology 82(1):335-345.
36. Mascarenhas J X, Korokhov N, Burger L, Kassim A, Tuter J, Miller D, Borgschulte T, George H J, Chang A, Pintel D J and others. 2017. Genetic engineering of CHO cells for viral resistance to minute virus of mice. Biotechnol Bioeng 114(3):576-588.
37. McNab F, Mayer-Barber K, Sher A, Wack A, O'Garra A. 2015. Type I interferons in infectious disease. Nature Reviews Immunology 15(2):87-103.
38. Merten O W. 2002. Virus contaminations of cell cultures—A biotechnological view. Cytotechnology 39(2):91-116.
39. Mutwiri G, Gerdts V, Lopez M, Babiuk L A. 2007. Innate immunity and new adjuvants. Rev Sci Tech 26(1):147-56.
40. Ng C S, Jogi M, Yoo J S, Onomoto K, Koike S, Iwasaki T, Yoneyama M, Kato H, Fujita T. 2013. Encephalomyocarditis Virus Disrupts Stress Granules, the Critical Platform for Triggering Antiviral Innate Immune Responses. Journal Of Virology 87(17):9511-9522.
41. Nims R W. 2006. Detection of adventitious viruses in biologicals—a rare occurrence. Dev Biol (Basel) 123:153-64; discussion 183-97.
42. Olive C. 2012. Pattern recognition receptors: sentinels in innate immunity and targets of new vaccine adjuvants. Expert Rev Vaccines 11(2):237-56.
43. Pantelic L, Sivakumaran H, Urosevic N. 2005. Differential induction of antiviral effects against West Nile virus in primary mouse macrophages derived from flavivirus-susceptible and congenic resistant mice by alpha/beta interferon and poly(I-C). J Virol 79(3):1753-64.
44. Perry A K, Chen G, Zheng D, Tang H, Cheng G. 2005. The host type I interferon response to viral and bacterial infections. Cell Res 15(6):407-22.
45. Pillai P S, Molony R D, Martinod K, Dong H, Pang I K, Tal M C, Solis A G, Bielecki P, Mohanty S, Trentalange M and others. 2016. Mx1 reveals innate pathways to antiviral resistance and lethal influenza disease. Science 352(6284):463-6.
46. Plant K P, Harbottle H, Thune R L. 2005. Poly I:C induces an antiviral state against Ictalurid Herpesvirus 1 and Mx1 transcription in the channel catfish (Ictalurus punctatus). Dev Comp Immunol 29(7):627-35.
47. Poiley J A, Nelson R E, Hillesund T, Rainer iR. 1991. Susceptibility of cho kl cells to infection by eight adventitious viruses and four retroviruses. In Vitro Toxicology 4(1):1-12.
48. Puig M, Tosh K W, Schramm L M, Grajkowska L T, Kirschman K D, Tami C, Beren J, Rabin R L, Verthelyi D. 2012. TLR9 and TLR7 agonists mediate distinct type I IFN responses in humans and nonhuman primates in vitro and in vivo. Journal Of Leukocyte Biology 91(1):147-158.
49. Rabenau H, Ohlinger V, Anderson J, Selb B, Cinatl J, Wolf W, Frost J, Mellor P, Doerr H W. 1993. Contamination of genetically engineered CHO-cells by epizootic haemorrhagic disease virus (EHDV). Biologicals 21(3):207-14.
50. Rieder M, Conzelmann K K. 2009. Rhabdovirus Evasion of the Interferon System. Journal Of Interferon And Cytokine Research 29(9):499-509.
51. Ronda, C., Pedersen, L. E., Hansen, H. G., Kallehauge, T. B. et al., Accelerating genome editing in CHO cells using CRISPR/Cas9 and CRISPy, a web-based target finding tool. Biotechnol. Bioeng. 2014, 111, 1604-1616.
52. Rupp O, MacDonald M L, Li S, Dhiman H, Polson S, Griep S, Heffner K, Hernandez I, Brinkrolf K, Jadhav V, Samoudi M, Hao H, Kingham B, Goesmann A, Betenbaugh M J, Lewis N E, Borth N, Lee K H. 2018. A reference genome of the Chinese hamster based on a hybrid assembly strategy. Biotechnol Bioeng. 115(8):2087-2100.
53. Sadler A J, Williams B R. 2008. Interferon-inducible antiviral effectors. Nat Rev Immunol 8(7):559-68.
54. Schneider W M, Chevillotte M D, Rice C M. 2014. Interferon-Stimulated Genes: A Complex Web of Host Defenses. Annual Review Of Immunology, Vol 32 32:513-545.
55. Schoggins J W, Rice C M. 2011. Interferon-stimulated genes and their antiviral effector functions. Curr Opin Virol 1(6):519-25.
56. Seo Y J, Hahm B. 2010. Type I interferon modulates the battle of host immune system against viruses. Adv Appl Microbiol 73:83-101.
57. Sharif-Askari E, Vassen L, Kosan C, Khandanpour C, Gaudreau M C, Heyd F, Okayama T, Jin J, Rojas M E, Grimes H L, Zeng H, Moroy T. 2010. Zinc finger protein Gfi1 controls the endotoxin-mediated Toll-like receptor inflammatory response by antagonizing NF-kappaB p65. Mol Cell Biol. 30(16):3929-42.
58. Sherry B. 2009. Rotavirus and Reovirus Modulation of the Interferon Response. Journal Of Interferon And Cytokine Research 29(9):559-567.
59. Subramanian A, Tamayo P, Mootha V K, Mukherjee S, Ebert B L, Gillette M A, Paulovich A, Pomeroy S L, Golub T R, Lander E S and others. 2005. Gene set enrichment analysis: a knowledge-based approach for interpreting genome-wide expression profiles. Proc Natl Acad Sci USA 102(43):15545-50.
60. Taber R, Alexander V, Wald N, Jr. 1976. The selection of virus-resistant Chinese hamster ovary cells. Cell 8(4):529-33.
61. Taniguchi T, Takaoka A. 2002. The interferon-alpha/beta system in antiviral responses: a multimodal machinery of gene regulation by the IRF family of transcription factors. Curr Opin Immunol 14(1):111-6.
62. Tisserand J, Khetchoumian K, Thibault C, Dembélé D, Chambon P, Losson R. 2011. Tripartite motif 24 (Trim24/Tif1α) tumor suppressor protein is a novel negative regulator of interferon (IFN)/signal transducers and activators of transcription (STAT) signaling pathway acting through retinoic acid receptor a (Rarα) inhibition. J Biol Chem. 286(38):33369-79.
63. Thompson A J, Locarnini S A. 2007. Toll-like receptors, RIG-I-like RNA helicases and the antiviral innate immune response. Immunol Cell Biol 85(6):435-45.
64. van Wijk X M, Dohrmann S, Hallstrom B M, Li S, Voldborg B G, Meng B X, McKee K K, van Kuppevelt T H, Yurchenco P D, Palsson B O and others. 2017. Whole-Genome Sequencing of Invasion-Resistant Cells Identifies Laminin alpha2 as a Host Factor for Bacterial Invasion. MBio 8(1).
65. Verhelst J, Hulpiau P, Saelens X. 2013. Mx proteins: antiviral gatekeepers that restrain the uninvited. Microbiol Mol Biol Rev 77(4):551-66.
66. Vishwanathan N, Bandyopadhyay A A, Fu H Y, Sharma M, Johnson K C, Mudge J, Ramaraj T, Onsongo G, Silverstein K A, Jacob N M and others. 2016. Augmenting Chinese hamster genome assembly by identifying regions of high confidence. Biotechnol J 11 (9): 1151-7.
67. Vishwanathan N, Yongky A, Johnson K C, Fu H Y, Jacob N M, Le H, Yusufi F N K, Lee D Y, Hu W S. 2015. Global Insights Into the Chinese Hamster and CHO Cell Transcriptomes. Biotechnology and Bioengineering 112 (5): 965-976.
68. Walsh D, Mathews M B, Mohr I. 2013. Tinkering with translation: protein synthesis in virus-infected cells. Cold Spring Harb Perspect Biol 5(1):a012351.
69. Walsh G. 2014. Biopharmaceutical benchmarks 2014. Nat Biotechnol 32(10):992-1000.
70. Wang Z, Gerstein M, Snyder M. 2009. RNA-Seq: a revolutionary tool for transcriptomics. Nat Rev Genet 10(1):57-63.
71. Weiebe M E, Becker F, Lazar R, May L, Casto B, Semense M, Fautz C, Garnick R, Miller C, Masover G and others. 1989. A multifaceted approach to assure that recombinant tPA is free of adventitious virus. In: Advances in animal cell biology and technology for bioprocesses. (Spier, Griffitlis, Stephenne, Crooy, eds.). 68-71.
72. Xu X, Nagarajan H, Lewis N E, Pan S, Cai Z, Liu X, Chen W, Xie M, Wang W, Hammond S and others. 2011. The genomic sequence of the Chinese hamster ovary (CHO)-K1 cell line. Nat Biotechnol 29(8):735-41.
73. Yuk I H, Zhang J D, Ebeling M, Berrera M, Gomez N, Werz S, Meiringer C, Shao Z, Swanberg J C, Lee K H and others. 2014. Effects of copper on CHO cells: insights from gene expression analyses. Biotechnol Prog 30(2):429-42.
74. Yusufi F N K, Lakshmanan M, Ho Y S, Loo B L W, Ariyaratne P, Yang Y, Ng S K, Tan T R M, Yeo H C, Lim H L, Ng S W, Hiu A P, Chow C P, Wan C, Chen S, Teo G, Song G, Chin J X, Ruan X, Sung K W K, Hu W S, Yap M G S, Bardor M, Nagarajan N, Lee D Y. 2017. Mammalian Systems Biotechnology Reveals Global Cellular Adaptations in a Recombinant CHO Cell Line. Cell Syst. 4(5):530-542.e6.

What is claimed is:

1. A method of inhibiting viral infection in a biological sample comprising administering to the sample an effective amount of:
a) a type I interferon or poly I:C;
b) a compound activating an innate immune response in the sample;
c) a compound suppressing expression of Gfi1, Trim24 and/or Cb1 in the sample; and/or
d) a compound activating expression of IRF7, IRF3, STAT1, STAT3, NFATC2, IRF5, STAT4, IRF9, IRF8, NFKB1, TP53, JUN and/or EBF1 in the sample.

2. The method of claim 1, wherein the biological sample is a cell culture.

3. The method of claim 1, wherein the biological sample comprises mammalian cells.

4. The method of claim 1, wherein the biological sample comprises CHO cells.

5. The method of claim 1, wherein the method is conducted in a biopharmaceutical manufacturing process.

6. The method of claim 1, wherein the compound suppresses expression of Gfi1, Trim24 and/or Cb1 in the sample.

7. The method of claim 1, wherein the compound activates expression of IRF7, IRF3, STAT1, STAT3, NFATC2, IRF5, STAT4, IRF9, IRF8, NFKB1, TP53, JUN and/or EBF1 in the sample.

8. The method of claim 1, wherein the virus is VSV, EMCV REO, an RNA virus, or a DNA virus.

9. The method of claim 1, wherein the compound is a nucleic acid.

10. A non-naturally occurring mammalian cell culture comprising cells genetically modified for suppressed expression of Gfi1, Trim24 and/or Cb1, and/or activated expression of IRF7, IRF3, STAT1, STAT3, NFATC2, IRF5, STAT4, IRF9, IRF8, NFKB1, TP53, JUN and/or EBF1, as compared to wild-type cells of the same mammalian species.

11. A method of producing a biopharmaceutical protein from a mammalian cell culture, comprising culturing mammalian cells having non-naturally occurring genetically suppressed expression of Gfi1, Trim24 and/or Cb1, and/or genetically activated expression of IRF7, IRF3, STAT1, STAT3, NFATC2, IRF5, STAT4, IRF9, IRF8, NFKB1, TP53, JUN and/or EBF1, as compared to wild-type cells of the same mammalian species; and isolating a protein of interest from the cultured cells.

12. The method of claim 11, wherein the biological sample comprises CHO cells.

13. The method of claim 11, wherein the cells have suppressed expression of Gfi1, Trim24 and/or Cb1.

14. The method of claim 11, wherein the cells have activated expression of IRF7, IRF3, STAT1, STAT3, NFATC2, IRF5, STAT4, IRF9, IRF8, NFKB1, TP53, JUN and/or EBF1.

15. A method of treating or preventing viral infection in a mammalian cell comprising administering to the cell an effective amount of:
a) a type I interferon or poly I:C;
b) a compound activating an innate immune response in the sample;
c) a compound suppressing expression of Gfi1, Trim24 or Cb1 in the sample; and/or
d) a compound activating expression of IRF7, IRF3, STAT1, STAT3, NFATC2, IRF5, STAT4, IRF9, IRF8, NFKB1, TP53, JUN and/or EBF1 in the sample.

16. A method for increasing virus infectivity in a mammalian cell comprising increasing expression of Gfi1, Trim24 or Cb1, and/or decreasing expression of IRF7, IRF3, STAT1, STAT3, NFATC2, IRF5, STAT4, IRF9, IRF8, NFKB1, TP53, JUN and/or EBF1 in the cell.

17. The method of claim 16, wherein the method further comprises isolating virus or viral particles from the cell.

18. The method of claim 16, wherein genetic material is delivered to the sample by viral transduction to increase or decrease expression of said gene.

19. A non-naturally occurring mammalian cell culture comprising mammalian cells having genetically activated expression of Gfi1, Trim24 or Cb1, and/or genetically suppressed expression of IRF7, IRF3, STAT1, STAT3, NFATC2, IRF5, STAT4, IRF9, IRF8, NFKB1, TP53, JUN and/or EBF1, as compared to wild-type cells of the same mammalian species.

20. A mammalian cell modified for in vivo suppressed expression or activity of Gfi1, Trim24 and/or Cb1, and/or activated expression or activity of IRF7, IRF3, STAT1, STAT3, NFATC2, IRF5, STAT4, IRF9, IRF8, NFKB1, TP53, JUN and/or EBF1, as compared to wild-type cells of the same mammalian species.
